SEQUENCE LISTING (34 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - protein, 25 aa, synthetic construct
EILEDMPVDPDNEAYEMPSEEGYQD

SEQ ID NO: 3 - DNA, 357 bp, synthetic construct
  source 1..357: mol_type = other DNA
gaggttcagctgcagcagtctggggcagagcttatgaagccaggggcctcagtcaagttgtcctgcacaggttctggcttcaacattaaagacacctatattcactgggtgaagcagaggcctgaacagggcctggagtggattggaaggattgatcctgcgaatagtaattctaaatatgacccgaggttccagggcaaggccactataacagcagacacatccgccaacacagcctacctgcagttcaccagcctgacatctgaggacactgccgtctattactgtactagacgctacgataatcggtacccgtttgcttactggggccaagggactctggtaactgtctctgca

SEQ ID NO: 4 - DNA, 336 bp, synthetic construct
  source 1..336: mol_type = other DNA
gatattgtgatgactcaggctgcaccctctgtacttgtcactcctggagagtcagtatccatctcctgcaggtctagtacgagtctcctgcatagtagtggcaagacttttttgtattggttcctgcagaggccaggccagtctcctcacctcctgatatatcggatgtccaaccttgcctcaggagtcccagacaggttcagtggcagtgggtcagggactgctttcacactgagaatcagtagagtggaggctgaggatgtgggtgtttattactgtatgcaacatctagagtatccgtacacgttcgggggggggaccaagctggaaataaaa

SEQ ID NO: 5 - protein, 119 aa, synthetic construct
EVQLQQSGAELMKPGASVKLSCTGSGFNIKDTYIHWVKQRPEQGLEWIGRIDPANSNSKYDPRFQGKATITADTSANTAYLQFTSLTSEDTAVYYCTRRYDNRYPFAYWGQGTLVTVSA

SEQ ID NO: 6 - protein, 112 aa, synthetic construct
DIVMTQAAPSVLVTPGESVSISCRSSTSLLHSSGKTFLYWFLQRPGQSPHLLIYRMSNLASGVPDRFSGSGSGTAFTLRISRVEAEDVGVYYCMQHLEYPYTFGGGTKLEIK

SEQ ID NO: 7 - protein, 5 aa, synthetic construct
DTYIH

SEQ ID NO: 8 - protein, 17 aa, synthetic construct
RIDPANSNSKYDPRFQG

SEQ ID NO: 9 - protein, 10 aa, synthetic construct
RYDNRYPFAY

SEQ ID NO: 10 - protein, 16 aa, synthetic construct
RSSTSLLHSSGKTFLY

SEQ ID NO: 11 - protein, 7 aa, synthetic construct
RMSNLAS

SEQ ID NO: 12 - protein, 9 aa, synthetic construct
MQHLEYPYT

SEQ ID NO: 13 - protein, 8 aa, synthetic construct
GFNIKDTY

SEQ ID NO: 14 - protein, 8 aa, synthetic construct
IDPANSNS

SEQ ID NO: 15 - protein, 12 aa, synthetic construct
TRRYDNRYPFAY

SEQ ID NO: 16 - protein, 11 aa, synthetic construct
TSLLHSSGKTF

SEQ ID NO: 17 - protein, 4 aa, synthetic construct
RMSN

SEQ ID NO: 18 - protein, 9 aa, synthetic construct
MQHLEYPYT

SEQ ID NO: 19 - DNA, 357 bp, synthetic construct
  source 1..357: mol_type = other DNA
gaagtacagcttattgagactggtggaggattggtgcagcctaaagggtcattaagactctcatgtgcagcctctggattcaccttcaataccaatgccatgacctgggtccgccaggctccaggacagggtttggaatgggttgctcgcataagaagtaaaattaataattatgcaacatattatgccgattcagtgaaagacaggttcaccatctccagagatgattcacaaagcatgctctatctacaaatgaacgacttgaaaactgaggactcagccatgtattactgtgtgaagactgggtactatgttatggactactggggtcaaggaacctcagtcaccgtctcctca

SEQ ID NO: 20 - DNA, 336 bp, synthetic construct
  source 1..336: mol_type = other DNA
aacattatgatgacacagtcgccatcatctctggctgtgtctccaggagaaaaggccactatgagctgtaagtccagtcaaagtgttttatacggttcaaatcagaaggactacttggcctggtatcagcagaagccaggacagtctcccaaactactgatctactgggcatccactagggaatctggtgtccctgatcgcttcacaggcagtggatctgggacagattttactcttaccatcagcagtgttcaagttgaagacctggcagtttattactgtcatcaataccgctcctcgtggacgttcggtggaggcaccaagctggaattcaaa

SEQ ID NO: 21 - protein, 119 aa, synthetic construct
EVQLIETGGGLVQPKGSLRLSCAASGFTFNTNAMTWVRQAPGQGLEWVARIRSKINNYATYYADSVKDRFTISRDDSQSMLYLQMNDLKTEDSAMYYCVKTGYYVMDYWGQGTSVTVSS

SEQ ID NO: 22 - protein, 112 aa, synthetic construct
NIMMTQSPSSLAVSPGEKATMSCKSSQSVLYGSNQKDYLAWYQQKPGQSPKLLIYWASTRESGVPDRFTGSGSGTDFTLTISSVQVEDLAVYYCHQYRSSWTFGGGTKLEFK

SEQ ID NO: 23 - protein, 5 aa, synthetic construct
TNAMT

SEQ ID NO: 24 - protein, 19 aa, synthetic construct
RIRSKINNYATYYADSVKD

SEQ ID NO: 25 - protein, 8 aa, synthetic construct
TGYYVMDY

SEQ ID NO: 26 - protein, 17 aa, synthetic construct
KSSQSVLYGSNQKDYLA

SEQ ID NO: 27 - protein, 7 aa, synthetic construct
WASTRES

SEQ ID NO: 28 - protein, 8 aa, synthetic construct
HQYRSSWT

SEQ ID NO: 29 - protein, 8 aa, synthetic construct
GFTFNTNA

SEQ ID NO: 30 - protein, 10 aa, synthetic construct
IRSKINNYAT

SEQ ID NO: 31 - protein, 10 aa, synthetic construct
VKTGYYVMDY

SEQ ID NO: 32 - protein, 12 aa, synthetic construct
QSVLYGSNQKDY

SEQ ID NO: 33 - protein, 4 aa, synthetic construct
WAST

SEQ ID NO: 34 - protein, 8 aa, synthetic construct
HQYRSSWT

(19) United States
(12) Patent Application Publication
(10) Pub. No.: US 2026/0337258 A1
(43) Pub. Date: Oct. 29, 2026

(54) ANTI-ALPHA-SYNUCLEIN ANTIBODIES AND USES THEREOF

(71) Applicant: UNIVERSITE LAVAL, Quebec (CA)

(72) Inventors: Martin LEVESQUE, Quebec (CA); Claude GRAVEL, Quebec (CA); Jean-Pierre JULIEN, Quebec (CA); Anne-Marie CASTONGUAY, Quebec (CA)

(21) Appl. No.: 19/527,910

(22) PCT Filed: Oct. 15, 2024
(86) PCT No.: PCT/CA2024/051353

§ 371 Date: Apr. 11, 2026

Related U.S. Application Data
(60) Provisional application No. 63/589,768, filed on Oct. 12, 2023.

(51) Int. Cl. C07K 16/18; A61K 31/711; A61K 35/17; A61K 38/00; A61K 40/11; A61K 40/15; A61K 40/31; A61P 25/28; C07K 14/705; C12N 5/0783; C12N 15/85; G01N 33/53

(52) U.S. Cl. CPC C07K 16/18; A61K 31/711; A61K 35/17; A61K 38/00; A61K 40/11; A61K 40/15; A61K 40/31; A61P 25/28; C07K 14/70503; C12N 5/0636; C12N 5/0646; C12N 15/85; G01N 33/53; C07K 2317/14; C07K 2317/24; C07K 2317/565; G01N 2800/2814; G01N 2800/52

(57) ABSTRACT

Described herein are anti-alpha-synuclein antibodies. More specifically, described herein are antibodies specific to serine 129 phosphorylated alpha-synuclein. Further described herein is the use of the anti alpha-synuclein antibodies for the treatment or diagnosis of a synucleinopathy. Further described are kits and compositions comprising the anti alpha-synuclein antibodies detecting alpha-synuclein in a sample.

The present description relates to anti-alpha-synuclein antibodies. More specifically, the present description relates to antibodies specific to serine 129-phosphorylated alpha-synuclein uses thereof for the treatment or diagnosis of a synucleinopathy.

The present description refers to a number of documents, the contents of which are herein incorporated by reference in their entirety.

### BACKGROUND

The exact mechanisms leading to the development and exacerbation of synucleinopathies, in particular Parkinson's disease (PD), are still unclear. These neurodegenerative diseases and disorders are characterised by the abnormal accumulation of α-synuclein (aSyn) aggregates in neurons, nerve fibres or glial cells. These aggregates are often referred to as Lewy bodies (LB). Although its pathological relevance is not quite clear, it is also accepted that phosphorylated aSyn, particularly at serine 129 (S129), is increased in patients with synucleinopathies. Antibodies recognizing aSyn, native and phosphorylated (e.g., at S129), have been previously described. However, there still remains a significant need for new therapies and diagnosis/prognosis methods of synucleinopathies. Furthermore, antibodies and agents that specifically recognize aSyn may be useful in the treatment and diagnosis/prognosis of synucleinopathies.

### SUMMARY

In some embodiments described herein, is an antibody or an antigen-binding fragment thereof that specifically binds to alpha-synuclein (aSyn), wherein the antibody or antigen-binding fragment thereof comprises:
a heavy chain variable region (VH) complementarity determining region 1 (CDR1), a VH CDR2, and a VH CDR3 from a VH comprising the amino acid sequence of SEQ ID NO: 5 or SEQ ID NO: 21; and
a light chain variable region (VL) CDR1, a VL CDR2, and a VL CDR3 from a VL comprising the amino acid sequence of SEQ ID NO: 6 or SEQ ID NO: 22.

In some embodiments described herein, is an antibody or an antigen-binding fragment thereof that specifically binds to alpha-synuclein (aSyn), wherein the antibody or antigen-binding fragment thereof comprises:
a heavy chain variable region (VH) comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 5 or SEQ ID NO: 21; and
a light chain variable region (VL) comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 6 or SEQ ID NO: 22.

In some embodiments described herein, is a polynucleotide encoding the antibody or antigen-binding fragment thereof defined herein.

In some embodiments described herein, is a vector comprising the polynucleotide defined herein.

In some embodiments described herein, is a cell comprising the polynucleotide defined herein or the vector defined herein.

In some embodiments described herein, is a chimeric antigen receptor (CAR) comprising the antibody or antigen-binding fragment thereof defined herein.

In some embodiments described herein, is a polypeptide comprising the antibody or antigen-binding fragment thereof defined herein.

In some embodiments described herein, is an in vitro method of making a cell expressing the antibody or antigen-binding fragment thereof defined herein comprising transducing the cell with the polynucleotide defined herein under suitable conditions.

In some embodiments described herein, is an in vitro method of making the antibody or antigen-binding fragment thereof defined herein comprising incubating the cell defined herein under suitable conditions.

In some embodiments described herein, is a pharmaceutical composition comprising the antibody or antigen-binding fragment thereof defined herein, the vector defined herein, the cell defined herein, the CAR defined herein, or the polypeptide defined herein, and a pharmaceutically acceptable carrier, excipient, diluent, solubilizer, emulsifier, preservative and/or adjuvant.

In some embodiments described herein, is a use of the antibody or antigen-binding fragment thereof defined herein, the vector defined herein, the cell defined herein, the CAR defined herein, the polypeptide defined herein, or the pharmaceutical composition defined herein in:
the treatment of a synucleinopathy;
the detection, diagnosis, or prognosis of a synucleinopathy; or
the monitoring of the progression of a synucleinopathy;
   in a subject.

In some embodiments described herein, is a use of the antibody or antigen-binding fragment thereof defined herein, the vector defined herein, the cell defined herein, the CAR defined herein, the polypeptide defined herein, or the pharmaceutical composition defined herein in the manufacture of a medicament for the treatment of a synucleinopathy.

In some embodiments described herein, is a method for treating a synucleinopathy in subject in need thereof, said method comprising administering the antibody or antigen-binding fragment thereof defined herein, the vector defined herein, the cell defined herein, the CAR defined herein, the polypeptide defined herein, or the pharmaceutical composition defined herein in said subject.

In some embodiments described herein, is a method for the detection, diagnosis, or prognosis of a synucleinopathy, or for the monitoring of the progression of a synucleinopathy, in a subject, said method comprising detecting alpha-synuclein in the subject or biological sample from said subject with the antibody or antigen-binding fragment thereof defined herein.

In some embodiments described herein, is an in vitro method of detecting alpha-synuclein (aSyn) in a sample, said method comprising:
contacting the sample with the antibody or antigen-binding fragment thereof defined herein to form a complex between the antibody or antigen-binding fragment thereof and alpha-synuclein;
detecting the complex between the antibody or antigen-binding fragment thereof and alpha-synuclein; and
optionally, measuring the levels of the complex between the antibody or antigen-binding fragment thereof and alpha-synuclein.

### BRIEF DESCRIPTION OF THE DRAWINGS

In the appended drawings:

FIG. 1A shows the 25 amino-acid peptide (SEQ ID NO: 2) corresponding to a sequence of alpha-Synuclein (human aSyn [Uniprot P37840-1]; SEQ ID NO: 1) close to the c-terminal, with a phosphorylated serine 129 (serine 129 in SEQ ID NO: 1, which is serine 19 in SEQ ID NO: 2) that was used to immunize mice to generate the monoclonal anti-aSyn antibodies. FIG. 1B shows the specificities of two different monoclonal antibodies obtained from this immunization (Mabs 833D4 and 73B3). Supernatants from the two hybridoma clones secreting these antibodies were used as primary antibodies on HEK (Human Embryonic Kidney) cells transfected with plasmid DNAs encoding either human aSyn alone (aSyn-WT), or human aSyn and PLK2 (Serine/threonine polo like kinase 2) (i.e., ser129 aSyn), or human aSyn S129G (serine 129 mutated to glycine) and PLK2, or an irrelevant protein (here Lmx1a-GFP), all under control of the Cytomegalovirus Immediate-Early promoter. Binding was demonstrated via Western Blot. FIG. 1C shows the immunohistofluorescence results of using each hybridoma supernatant as primary antibodies in immunofluorescence on the four (4) different populations of transfected cells, as described in FIG. 1B. FIG. 1D shows the analysis of the binding of the Mab 833D4 to either monomers (M), oligomers (O), or fibrils (F) of human aSyn by dot immunobinding assay. BSA was used as a negative control.

FIG. 2 shows the binding of two monoclonal anti-aSyn antibodies (833D4 and 73B3) to pathological aggregates in human brain sections using indirect immunofluorescence. The two monoclonal IgG antibodies were purified from the hybridoma culture media using Protein G agarose beads and were tested on human PD (Parkinson's disease) and control brain sections (age-matched) to detect pathological aSyn.

FIG. 3A shows the structure of scFvs which were generated by assembling the cDNA sequences encoding the variable region of the light (Vk or VL) and the heavy chain (VH) of the monoclonal antibody into an open reading frame linked by a DNA sequence encoding a short, flexible peptide (multiples of Gly-(Ser)4), and cloning into an expression plasmid. FIG. 3B upper right shows the structure of the expression plasmid containing sequences encoding a CMV enhancer/promoter, a SV-40 intron, the scFv open reading frame, and a length-optimized Woodchuck Hepatitis Virus Posttranscriptional Regulatory Element coupled to a Simian Virus 40 polyadenylation signal for increased expression (that we termed WPpA) (Choi, J-H. et al., 2014). A sequence encoding the human c-Myc tag was added at the c-terminal of one of the variable regions in order to facilitate detection of the expressed protein. Expression plasmids containing the VH/VL pairings of the two clones (833D4 and 73B3) were generated. FIG. 3B lower right shows a schematic of an AAV viral genome obtained after co-transfection of one such scFv plasmid vector and AAV helper plasmids into HEK cells. FIG. 3C shows the dot blot analysis of the binding of scFvs contained in the culture media from HEK cells transfected with each of either the scFv expression plasmids encoding the 73B3, or 833D4, or encoding a control scFv (derived from an anti-GFP antibody), on purified human aSyn protein and purified phosphorylated S129-aSyn protein. Bovine Serum Albumin (BSA) was used as a negative protein control. FIG. 3D shows the ELISA analysis of the binding of scFvs contained in culture media from HEK cells transfected with scFv expression plasmids on purified human aSyn protein and phosphorylated S129-aSyn protein (pS129-aSyn or paSyn). BSA was used as a negative control. Commercially available antibodies CST-2628 (Cell Signaling Technology™ and anti-Phospho α-Synuclein Ser129 antibody (pSyn #64; FUJIFILM™ Wako Pure Chemical Corporation) were used as positive controls for the ELISA (data not shown).

FIG. 4A shows the schematic protocol for the systemic administration of scFvs in an aggressive synucleinopathy mouse model (Luk et al., 2012). The self-complementary Adeno-Associated Viruses encoding the scFv-73B3, scFv-833D4, or control scFv, and bearing the Cap-B10 capsid were generated by co-transfection of HEK-293 cells with the proper AAV-packaging plasmids, and purified virus solutions were administered intravenously in the retro-orbital sinus 7 days after injection of preformed human aSyn fibrils (PFFs) in the dorsal striatum. Starting 8 weeks after PFF (or buffer for control) injection, mice were closely monitored with a scoring system to detect any motor impairment. FIG. 4B shows the clinical score around the time of sacrifice (days 82-84 after PFF injection) for the mice that received the scAAV encoding the control scFv, or no scFv, compared to the ones injected with the scAAVs encoding either scFv-833D4 or scFv-73B3 one week after PFF injection. FIGS. 4C and 4D show the results of the grip strength test and the rotarod test respectively at 12 weeks after PFF (or PBS) injection in the same mice. FIG. 4E shows the presence of the scFv-73B3 in the cortex, thalamus, midbrain, and medulla regions of the brain of mice 12 weeks after intravenous injection of a scFv-73B3-encoding AAV vector bearing the CapB10 capsid, as seen following the immunofluorescence detection of the human cMyc epitope present at the carboxy end of the scFv proteins.

FIGS. 5A, 5B, and 5C illustrate the distribution of aggregates of pS129-aSyn observed in the hypothalamus, midbrain, and medulla, respectively, of the mice groups described in FIG. 4A following sacrifice. FIG. 5D shows representative microphotographs images showing by immunofluorescence (white dots) the presence of phosphorylated S129-aSyn aggregates—in sections of the hypothalamus, midbrain, and medulla of the PFF-injected mice that were administered one week later the viral vectors encoding either the control scFv, the scFv-73B3, or the scFv-833D4, and sacrificed 12 weeks after viral vector administration.

FIG. 6A shows a Western Blot analysis of total aSyn and pS129-aSyn in brain extracts from mice following treatment with PFF/PBS and the scFvs, as described in FIG. 4A. FIGS. 6B and 6C show the quantification of pS129-aSyn normalized to either total aSyn or actin, respectively, via densitometry. FIG. 6D shows the quantification of total aSyn normalized to actin via densitometry. PFF (n=4); PBS (n=3).

FIGS. 7A-7E shows the binding affinity of antibodies 833D4 and 73B3 to alpha-synuclein monomers and phosphorylated monomers (pS129-aSyn). FIGS. 7A-7D shows the results of surface plasmon resonance (SPR) real time binding curves for different concentrations of Mab 833D4 and 73B3 towards WT alpha-synuclein monomers and phosphorylated monomers (pS129-aSyn). FIG. 7E shows the Kinetic values measured with the SPR experiment (Ka=association constant, Kd=dissociation constant, KD=Equilibrium constant)

FIGS. 8A-8E shows the prevention of alpha-synuclein aggregation with antibodies 833D4 and 73B3 in vitro. FIG. 8A shows the results of a Thioflavin T (ThT) aggregation assay performed by incubating alpha-synuclein monomers at 37 °C with agitation for 5 days with or without addition of monoclonal antibodies 833D4 or a commercial anti-aSyn antibody from Abcam (Abcam ab209420; Clone MJFR1) in the reaction mix. FIGS. 8B-8C show the results of a Real-Time Quaking-Induced Conversion (RT-QuIC) assay where monoclonal antibody 833D4 is incubated with agitation in combination with Dementia with Lewy bodies (DLB) (FIG. 8B) or control (FIG. 8C) brain homogenates. Alpha-synuclein aggregation is measured with ThT. FIG. 8D shows a comparison of phosphorylated aSyn (pS129) staining with clone 73B3 with a commercial anti-Phospho α-Synuclein Ser129 antibody from Wako (pSyn #64; FUJIFILM™ Wako Pure Chemical Corporation) in brain sections from PD mice and untreated mice (scale bar=50 μm). PD mice were induced by injection of aSyn PFFs into the dorsal striatum of M83 mice (transgenic mice overexpressing human aSyn A53T). FIG. 8E shows a comparison of phosphorylated aSyn (pS129) staining with clone 73B3 and a commercial anti-alpha-synuclein (phospho S129) antibody from Abcam (ab51253) in primary cultures of midbrain neurons with or without addition of aSyn preformed fibrils (PFF) (scale bar=100 μm).

FIGS. 9A-9B show the prevention of alpha-synuclein fibril internalization in human neurons by 833D4 and 73B3 scFvs. Neurons were generated from human induced pluripotent stem cells (iPSCs). The neurons were infected with AAV-scFv 833D4 or 73B3 and then exposed for 2 h to alpha-synuclein preformed fibrils (PFF) tagged with AlexaFluor™ 488. FIG. 9A shows representative images of dopaminergic (TH+) neurons expressing the scFvs (Myc+) and the intracellular 488-PFF. FIG. 9B shows the quantification of the intensity of 488-PFF inside dopaminergic neurons (corrected total cell fluorescence, CTCF). One-Way ANOVA with Dunnett's multiple comparison test.

FIGS. 10A-10D show the internalization of 833D4-scFv by neurons and microglia. FIG. 10A shows a schematic of the experiment for the expression of scFv-secreting and non-secreting cells. Cells that are both HA and GFP positive have been infected by the vector and are secreting cells. Cells that are HA positive, but GFP negative were not infected by the vector and have internalized the 833D4-scFv. FIG. 10B shows fluorescence microscopy images taken in the medulla showing non-secreting cells (white arrows) that have internalized the 833D4-scFv. FIG. 10C shows fluorescence microscopy images showing the colocalization of the 833D4-scFv with pS129-aSyn inside a neuron. FIG. 10D shows fluorescence microscopy images showing the internalization of the 833D4-scFv by microglia (white arrows).

FIGS. 11A-11C show the safety results of AAV-scFv systemic injection. Transgenic mice expressing a luciferase reporter under the control of the TLR2 promoter were used. TLR2 receptor is induced in inflammatory conditions. The level of luminescence is proportional to the level of TLR2, an indirect measure of inflammation. Mice were injected intravenously with LPS (positive control), saline, AAV-GFP, AAV-scFv-control, AAV-scFv-73B3 or AAV-scFv-833D4. FIG. 11A shows representative images of the bioluminescence signal in injected mice. FIG. 11B shows total flux (photon/second) of the bioluminescence signal in each group from baseline to 30 days post-injection. FIG. 11C shows total flux at 4 days post-injection. N=3 mice/group.

### SEQUENCE LISTING

This application contains a Sequence Listing in computer readable form created Oct. 6, 2024. The computer readable form is incorporated herein by reference.

TABLE 1
Sequence Listing

| SEQ ID NO: | Description |
|---|---|
| 1 | Human alpha-Synuclein (Uniprot P37840-1) |
| 2 | C-terminal aSyn peptide |
| 3 | 73B3 VH DNA Sequence |
| 4 | 73B3 VL DNA Sequence |
| 5 | 73B3 VH AA Sequence |
| 6 | 73B3 VL AA Sequence |
| 7 | 73B3 VH CDR1 (Kabat) |
| 8 | 73B3 VH CDR2 (Kabat) |
| 9 | 73B3 VH CDR3 (Kabat) |
| 10 | 73B3 VL CDR1 (Kabat) |
| 11 | 73B3 VL CDR2 (Kabat) |
| 12 | 73B3 VL CDR3 (Kabat) |
| 13 | 73B3 VH CDR1 (IMGT) |
| 14 | 73B3 VH CDR2 (IMGT) |
| 15 | 73B3 VH CDR3 (IMGT) |
| 16 | 73B3 VL CDR1 (IMGT) |
| 17 | 73B3 VL CDR2 (IMGT |
| 18 | 73B3 VL CDR3 (IMGT) |
| 19 | 833D4 VH DNA Sequence |
| 20 | 833D4 VL DNA Sequence |
| 21 | 833D4 VH AA Sequence |
| 22 | 833D4 VL AA Sequence |
| 23 | 833D4 VH CDR1 (Kabat) |
| 24 | 833D4 VH CDR2 (Kabat) |
| 25 | 833D4 VH CDR3 (Kabat) |
| 26 | 833D4 VL CDR1 (Kabat) |
| 27 | 833D4 VL CDR2 (Kabat) |
| 28 | 833D4 VL CDR3 (Kabat) |
| 29 | 833D4 VH CDR1 (IMGT) |
| 30 | 833D4 VH CDR2 (IMGT) |
| 31 | 833D4 VH CDR3 (IMGT) |
| 32 | 833D4 VL CDR1 (IMGT) |
| 33 | 833D4 VL CDR2 (IMGT) |
| 34 | 833D4 VL CDR3 (IMGT) |

### DETAILED DESCRIPTION

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about”, when used herein, indicates that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9, and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, “protein” or “polypeptide” or “peptide” means any peptide-linked chain of amino acids, which may or may not comprise any type of modification (e.g., chemical or post-translational modifications such as acetylation, carboxymethylation, deamidation, phosphorylation, citrullination, homocitrullination, glycosylation, oxidation, sulfatation, sumoylation, prenylation, ubiquitination, etc.).

As used herein, “subject” refers to a mammal, including but not limited to a human, mouse, rat, or rabbit. The subject may be a human patient, such as a patient having or suspected of having a synucleinopathy. The subject may also be a “healthy”, “reference” (e.g., suitable reference), or “control” subject, which may not suffer from having a synucleinopathy or does not present symptoms associated with having a synucleinopathy and may be a healthy subject, which may be used as a control or reference in the determination of the presence, diagnosis, prognosis, classification, or progression of having a synucleinopathy.

As used herein, “synucleinopathy”, also referred to as “alpha-synucleinopathy (α-synucleinopathy)” refers to a neurodegenerative disease or disorder characterised by the abnormal accumulation of α-synuclein aggregates in neurons, nerve fibres or glial cells of a subject. Parkinson's disease (PD), Lewy body disease (LBD), dementia with Lewy bodies, Multiple systemic atrophy (MSA), pure autonomic failure, or rapid eye movement (REM) sleep behaviour disorder, Parkinson's disease (PD).

As used herein, “sample” or “biological sample” refers to any sample comprising or being tested for the presence of alpha-synuclein or phosphorylated alpha-synuclein. Samples can include but are not limited to cellular extracts, live or dead/dying cells, cell lysates, extracellular fluid, fluid harvested from the body of a subject, culture media, blood, bone marrow, plasma, serum, sputum, bronchoalveolar lavage (BAL), synovial fluid, cerebral spinal fluid, biopsy (e.g., brain biopsy), or any organ.

As used herein the expression “corresponding”, “positionally equivalent” or “homologous” refer to mutations in two or more different aSyn genes or proteins (e.g., from the same or different organism, or different variants, isoforms, homologs, analogs, orthologs, or paralogs) occur at homologous amino acid positions in the amino acid sequences of the aSyn. Hence, use herein of the term “positionally equivalent” or “homologous” to a given mutation, regardless of whether or not the particular function of the mutated amino acid is known. It is possible to identify positionally equivalent or homologous amino acid residues in the amino acid sequences of two or more different aSyn genes or proteins on the basis of sequence alignment and/or molecular modelling.

As used herein, the term “antibody” refers to an immunoglobulin which binds specifically to an antigen. In general, and antibody can comprise at least two heavy (H) chains and two light (L) chains interconnected by disulfide bonds, or an antigen-binding fragment thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs may mediate the binding of the immunoglobulin to host tissues or factors. Antigen-binding fragments of antibodies may include any one or a combination of the components described herein.

Antibodies and antigen-binding fragments thereof may include, for example, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), scFv-Fc, camelized antibodies, affybodies, Fab fragments, F(ab′)₂ fragments, disulfide-linked Fvs (sdFv), anti-idiotypic (anti-Id) antibodies (including, e.g., anti-anti-Id antibodies), minibodies, domain antibodies, a kappa-lambda body, a dual-affinity re-targeting molecule (DART), a knob-in-hole, a strand-exchange engineered domain body (SEEDbody), a Bispecific T cell engager (BiTe), a CrossMab, an Fcab, a Diabody, a Tandem diabody (TandAb), and a DuoBody, synthetic antibodies (sometimes referred to herein as “antibody mimetics”), and antigen-binding fragments of any of the above. In certain embodiments, antibodies described herein refer to polyclonal antibody populations.

As used herein, the term “heavy chain” when used in reference to an antibody can refer to any distinct type, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG2a/2c, IgG2b, IgG3, or IgG4.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

As used herein, the terms “constant region” and “constant domain” are interchangeable and have a meaning common in the art. The constant region is an antibody portion, e.g., a carboxyl terminal portion of a light and/or heavy chain which is not directly involved in binding of an antibody to the antigen, but which can exhibit various effector functions, such as interaction with the Fc receptor. The constant region of an immunoglobulin molecule generally has a more conserved amino acid sequence relative to an immunoglobulin variable domain. The antibodies or antigen-binding fragments thereof defined herein do not necessarily comprise a specific heavy chain and/or light chain constant domain.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs) (e.g., humanized antibodies).

A number of definitions of the CDRs are commonly in use: Kabat numbering, IMGT numbering, Chothia numbering, AbM numbering, or contact numbering. The AbM definition is a compromise between the two used by Oxford Molecular's AbM antibody modelling software. The contact definition is based on an analysis of the available complex crystal structures. The skilled person in the art that the CDR sequences can be determined from a given VH and VL sequence using these established numbering systems. Exemplary VH and VL CDRs are defined further below using the Kabat and IMGT numbering systems. However, the present description also relates to VH and VL CDRs from the antibody clones defined herein using any numbering system.

As used herein, the terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art.

As used herein, the term “administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intracranial, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

As used herein, the term “chimeric antigen receptor (CAR)” refers to a receptor comprising the VH and VL or one or more VH and/or VL CDRs from an antibody, which is then expressed (e.g., via known engineering methods) on the surface of a cell. In some embodiments, the cell is an immune cell (e.g., CAR T cell or CAR NK cell). The cell may be from a subject. In some embodiments, the CAR comprises structural elements known in the art such that the CAR is functionally active upon recognition with the corresponding antigen. For example, the CAR may include an activation domain, a stimulation domain, stimulatory domain, costimulatory domain, and any combination thereof. Examples of said structural elements are known in the art.

The present description relates to an antibody or an antigen-binding fragment thereof that specifically binds to alpha-synuclein (aSyn) (e.g., phosphorylated and/or non-phosphorylated aSyn). In one aspect, the present description relates to an antibody or an antigen-binding fragment thereof that specifically binds phosphorylated aSyn. In one aspect, the present description relates to an antibody or an antigen-binding fragment thereof that has higher binding affinity for phosphorylated aSyn (e.g., pS129-aSyn) relative to unphosphorylated aSyn. In one aspect, the present description relates to an antibody or an antigen-binding fragment thereof that preferentially binds to phosphorylated aSyn (e.g., pS129-aSyn) relative to unphosphorylated aSyn. In one aspect, the present description relates to an antibody or an antigen-binding fragment thereof having higher binding affinity for aSyn oligomers than for aSyn monomers and/or aSyn fibrils. The antibody or antigen-binding fragment thereof comprises a heavy chain variable region (VH) comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 5 or SEQ ID NO: 21; and a light chain variable region (VL) comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 6 or SEQ ID NO: 22.

Exemplary VH and VL nucleotide and amino acid sequences from two clones, 73B3 and 833D4, are shown in Table 2.

TABLE 2
VH and VL sequences of 73B3 and 833D4

| Anti-aSyn | | |
|---|---|---|
| Antibody | | |
| Clone | Nucleotide Sequence | Amino Acid Sequence |
| 73B3 VH | GAGGTTCAGCTGCAGCAGTCTGGGGCA | EVQLQQSGAELMKPGASVKLS |
| | GAGCTTATGAAGCCAGGGGCCTCAGTC | CTGSGFNIKDTYIHWVKQRPEQ |
| | AAGTTGTCCTGCACAGGTTCTGGCTTC | GLEWIGRIDPANSNSKYDPRFQ |
| | AACATTAAAGACACCTATATTCACTGG | GKATITADTSANTAYLQFTSLTS |
| | GTGAAGCAGAGGCCTGAACAGGGCCT | EDTAVYYCTRRYDNRYPFAYW |
| | GGAGTGGATTGGAAGGATTGATCCTGC | GQGTLVTVSA (SEQ ID NO: 5) |
| | GAATAGTAATTCTAAATATGACCCGAG | |
| | GTTCCAGGGCAAGGCCACTATAACAGC | |
| | AGACACATCCGCCAACACAGCCTACCT | |
| | GCAGTTCACCAGCCTGACATCTGAGGA | |
| | CACTGCCGTCTATTACTGTACTAGACG | |
| | CTACGATAATCGGTACCCGTTTGCTTA | |
| | CTGGGGCCAAGGGACTCTGGTAACTGT | |
| | CTCTGCA (SEQ ID NO: 3) | |
| 73B3 VL | GATATTGTGATGACTCAGGCTGCACCC | DIVMTQAAPSVLVTPGESVSISC |
| | TCTGTACTTGTCACTCCTGGAGAGTCA | RSSTSLLHSSGKTFLYWFLQRP |
| | GTATCCATCTCCTGCAGGTCTAGTACG | GQSPHLLIYRMSNLASGVPD |
| | AGTCTCCTGCATAGTAGTGGCAAGACT | RFSGSGSGTAFTLRISRVEAEDV |
| | TTTTTGTATTGGTTCCTGCAGAGGCCAG | GVYYCMQHLEYPYTFGGGTKL |
| | GCCAGTCTCCTCACCTCCTGATATATCG | EIK (SEQ ID NO: 6) |
| | GATGTCCAACCTTGCCTCAGGAGTCCC | |
| | AGACAGGTTCAGTGGCAGTGGGTCAGG | |
| | GACTGCTTTCACACTGAGAATCAGTAG | |
| | AGTGGAGGCTGAGGATGTGGGTGTTTA | |
| | TTACTGTATGCAACATCTAGAGTATCC | |
| | GTACACGTTCGGGGGGGGGACCAAGCT | |
| | GGAAATAAAA (SEQ ID NO: 4) | |
| 833D4 VH | GAAGTACAGCTTATTGAGACTGGTGGA | EVQLIETGGGLVQPKGSLRLSC |
| | GGATTGGTGCAGCCTAAAGGGTCATTA | AASGFTFNTNAMTWVRQAPGQ |
| | AGACTCTCATGTGCAGCCTCTGGATTC | GLEWVARIRSKINNYATYYADS |
| | ACCTTCAATACCAATGCCATGACCTGG | VKDRFTISRDDSQSMLYLQMN |
| | GTCCGCCAGGCTCCAGGACAGGGTTTG | DLKTEDSAMYYCVKTGYYVM |
| | GAATGGGTTGCTCGCATAAGAAGTAAA | DYWGQGTSVTVSS (SEQ ID |
| | ATTAATAATTATGCAACATATTATGCC | NO: 21) |
| | GATTCAGTGAAAGACAGGTTCACCATC | |
| | TCCAGAGATGATTCACAAAGCATGCTC | |
| | TATCTACAAATGAACGACTTGAAAACT | |
| | GAGGACTCAGCCATGTATTACTGTGTG | |
| | AAGACTGGGTACTATGTTATGGACTAC | |
| | TGGGGTCAAGGAACCTCAGTCACCGTC | |
| | TCCTCA (SEQ ID NO: 19) | |
| 833D4 VL | AACATTATGATGACACAGTCGCCATCA | NIMMTQSPSSLAVSPGEKATMS |
| | TCTCTGGCTGTGTCTCCAGGAGAAAAG | CKSSQSVLYGSNQKDYLAWYQ |
| | GCCACTATGAGCTGTAAGTCCAGTCAA | QKPGQSPKLLIYWASTRESGVP |
| | AGTGTTTTATACGGTTCAAATCAGAAG | DRFTGSGSGTDFTLTISSVQVED |
| | GACTACTTGGCCTGGTATCAGCAGAAG | LAVYYCHQYRSSWTFGGGTKL |
| | CCAGGACAGTCTCCCAAACTACTGATC | EFK (SEQ ID NO: 22) |
| | TACTGGGCATCCACTAGGGAATCTGGT | |
| | GTCCCTGATCGCTTCACAGGCAGTGGA | |
| | TCTGGGACAGATTTTACTCTTACCATCA | |
| | GCAGTGTTCAAGTTGAAGACCTGGCAG | |
| | TTTATTACTGTCATCAATACCGCTCCTC | |
| | GTGGACGTTCGGTGGAGGCACCAAGCT | |
| | GGAATTCAAA (SEQ ID NO: 20) | |

In some aspects, the sequence variation occurs outside of the CDR sequences. In some aspects, the sequence identity is over the full length of the defined sequence (e.g., VH/VL).

The antibody or antigen-binding fragment thereof comprises a heavy chain variable region (VH) complementarity determining region 1 (CDR1), a VH CDR2, and a VH CDR3 from a VH comprising the amino acid sequence of SEQ ID NO: 5 or SEQ ID NO: 21; and a light chain variable region (VL) CDR1, a VL CDR2, and a VL CDR3 from a VL comprising the amino acid sequence of SEQ ID NO: 6 or SEQ ID NO: 22. Exemplary VH and VL CDRs are defined in Table 3 using the Kabat and IMGT numbering systems. However, the present description also relates to VH and VL CDRs from the antibody clones defined herein using any numbering system.

TABLE 2
VH and VL CDR sequences of 73B3 and 833D4

| Anti-aSyn | | | |
|---|---|---|---|
| Antibody | | | |
| Clone | Amino Acid Sequence | Amino Acid Sequence | CDR |
| 73B3 VH | EVQLQQSGAELMKPGASVKL | DTYIH | CDR1 |
| CDRs | SCTGSGFNIKDTYIHWVKQRP | (SEQ ID NO: 7) | |
| (Bolded and | EQGLEWIGRIDPANSNSKYDP | RIDPANSNSKYDPRFQG | CDR2 |
| underlined; | RFQGKATITADTSANTAYLQ | (SEQ ID NO: 8) | |
| Kabat) | FTSLTSEDTAVYYCTRRYDN | RYDNRYPFAY | CDR3 |
| | RYPFAYWGQGTLVTVSA | (SEQ ID NO: 9) | |
| | (SEQ ID NO: 3) | | |
| 73B3 VL | DIVMTQAAPSVLVTPGESVSIS | RSSTSLLHSSGKTFLY | CDR1 |
| CDRs | CRSSTSLLHSSGKTFLYWFL | (SEQ ID NO: 10) | |
| (Bolded and | QRPGQSPHLLIYRMSNLASGV | RMSNLAS | CDR2 |
| underlined; | PDRFSGSGSGTAFTLRISRVEA | (SEQ ID NO: 11) | |
| Kabat) | EDVGVYYCMQHLEYPYTFG | MQHLEYPYT | CDR3 |
| | GGTKLEIK (SEQ ID NO: 4) | (SEQ ID NO: 12) | |
| 73B3 VH | EVQLQQSGAELMKPGASVKL | GFNIKDTY | CDR1 |
| CDRs | SCTGSGFNIKDTYIHWVKQR | (SEQ ID NO: 13) | |
| (Bolded and | PEQGLEWIGRIDPANSNSKYD | IDPANSNS | CDR2 |
| underlined; | PRFQGKATITADTSANTAYLQ | (SEQ ID NO: 14) | |
| IMGT) | FTSLTSEDTAVYYCTRRYDNR | TRRYDNRYPFAY | CDR3 |
| | YPFAYWGQGTLVTVSA (SEQ | (SEQ ID NO: 15) | |
| | ID NO: 3) | | |
| 73B3 VL | DIVMTQAAPSVLVTPGESVSIS | TSLLHSSGKTF | CDR1 |
| CDRs | CRSSTSLLHSSGKTFLYWFL | (SEQ ID NO: 16) | |
| (Bolded and | QRPGQSPHLLIYRMSNLASGV | RMS or RMSN | CDR2 |
| underlined; | PDRFSGSGSGTAFTLRISRVEA | (SEQ ID NO: 17) | |
| IMGT) | EDVGVYYCMQHLEYPYTFG | MQHLEYPYT | CDR3 |
| | GGTKLEIK (SEQ ID NO: 4) | (SEQ ID NO: 18) | |
| 833D4 VH | EVQLIETGGGLVQPKGSLRLS | TNAMT | CDR1 |
| CDRs | CAASGFTFNTNAMTWVRQA | (SEQ ID NO: 23) | |
| (Bolded and | PGQGLEWVARIRSKINNYAT | RIRSKINNYATYYADSVKD | CDR2 |
| underlined; | YYADSVKDRFTISRDDSQSML | (SEQ ID NO: 24) | |
| Kabat) | YLQMNDLKTEDSAMYYCVK | TGYYVMDY | CDR3 |
| | TGYYVMDYWGQGTSVTVSS | (SEQ ID NO: 25) | |
| | (SEQ ID NO: 21) | | |
| 833D4 VL | NIMMTQSPSSLAVSPGEKATM | KSSQSVLYGSNQKDYLA | CDR1 |
| CDRs | SCKSSQSVLYGSNQKDYLA | (SEQ ID NO: 26) | |
| (Bolded and | WYQQKPGQSPKLLIYWASTR | WASTRES | CDR2 |
| underlined; | ESGVPDRFTGSGSGTDFTLTIS | (SEQ ID NO: 27) | |
| Kabat) | SVQVEDLAVYYCHQYRSSW | HQYRSSWT | CDR3 |
| | TFGGGTKLEFK (SEQ ID NO: | (SEQ ID NO: 28) | |
| | 22) | | |
| 833D4 VH | EVQLIETGGGLVQPKGSLRLS | GFTFNTNA | CDR1 |
| CDRs | CAASGFTFNTNAMTWVRQA | (SEQ ID NO: 29) | |
| (Bolded and | PGQGLEWVARIRSKINNYAT | IRSKINNYAT | CDR2 |
| underlined; | YYADSVKDRFTISRDDSQSML | (SEQ ID NO: 30) | |
| IMGT) | YLQMNDLKTEDSAMYYCVK | VKTGYYVMDY | CDR3 |
| | TGYYVMDYWGQGTSVTVSS | (SEQ ID NO: 31) | |
| | (SEQ ID NO: 21) | | |
| 833D4 VL | NIMMTQSPSSLAVSPGEKATM | QSVLYGSNQKDY | CDR1 |
| CDRs | SCKSSQSVLYGSNQKDYLAW | (SEQ ID NO: 32) | |
| (Bolded and | YQQKPGQSPKLLIYWASTRES | WAS or WAST | CDR2 |
| underlined; | GVPDRFTGSGSGTDFTLTISSV | (SEQ ID NO: 33) | |
| IMGT) | QVEDLAVYYCHQYRSSWTF | HQYRSSWT | CDR3 |
| | GGGTKLEFK (SEQ ID NO: 22) | (SEQ ID NO: 34) | |

In some aspects, the antibody or antigen-binding fragment thereof comprises a VH CDR1, a VH CDR2, and a VH CDR3 from a VH comprising the amino acid sequence of SEQ ID NO: 5, and a VL CDR1, a VL CDR2, and a VL CDR3 from the VL comprising the amino acid sequence of SEQ ID NO: 6.

In some aspects, the antibody or antigen-binding fragment thereof comprises a VH CDR1, a VH CDR2, and a VH CDR3 from a VH comprising the amino acid sequence of SEQ ID NO: 21, and a VL CDR1, a VL CDR2, and a VL CDR3 from the VL comprising the amino acid sequence of SEQ ID NO: 22.

In some aspects, the antibody or antigen-binding fragment thereof comprises a VH CDR1 comprising the amino acid sequence of SEQ ID NO: 7, a VH CDR2 comprising the amino acid sequence of SEQ ID NO: 8, a VH CDR3 comprising the amino acid sequence of SEQ ID NO: 9, a VL CDR1 comprising the amino acid sequence of SEQ ID NO: 10, a VL CDR2 comprising the amino acid sequence of SEQ ID NO: 11, and a VL CDR3 comprising the amino acid sequence of SEQ ID NO: 12.

In some aspects, the antibody or antigen-binding fragment thereof comprises a VH CDR1 comprising the amino acid sequence of SEQ ID NO: 13, a VH CDR2 comprising the amino acid sequence of SEQ ID NO: 14, a VH CDR3 comprising the amino acid sequence of SEQ ID NO: 15, a VL CDR1 comprising the amino acid sequence of SEQ ID NO: 16, a VL CDR2 comprising the amino acid sequence RMS or the amino acid sequence of SEQ ID NO: 17, and a VL CDR3 comprising the amino acid sequence of SEQ ID NO: 18.

In some aspects, the antibody or antigen-binding fragment thereof comprises a VH CDR1 comprising the amino acid sequence of SEQ ID NO: 23, a VH CDR2 comprising the amino acid sequence of SEQ ID NO: 24, a VH CDR3 comprising the amino acid sequence of SEQ ID NO: 25, a VL CDR1 comprising the amino acid sequence of SEQ ID NO: 26, a VL CDR2 comprising the amino acid sequence of SEQ ID NO: 27, and a VL CDR3 comprising the amino acid sequence of SEQ ID NO: 28.

In some aspects, the antibody or antigen-binding fragment thereof comprises a VH CDR1 comprising the amino acid sequence of SEQ ID NO: 29, a VH CDR2 comprising the amino acid sequence of SEQ ID NO: 30, a VH CDR3 comprising the amino acid sequence of SEQ ID NO: 31, a VL CDR1 comprising the amino acid sequence of SEQ ID NO: 32, a VL CDR2 comprising the amino acid sequence WAS or the amino acid sequence of SEQ ID NO: 33, and a VL CDR3 comprising the amino acid sequence of SEQ ID NO: 34.

In some aspects, the antibody or antigen-binding fragment thereof comprises a VH comprising an amino acid sequence that is at least about 70%, at least about 75%, at least about 80%, at least about 85%, at least about 90%, at least about 95%, at least about 96%, at least about 97%, at least about 98%, at least about 99% identical to SEQ ID NO: 5 or SEQ ID NO: 21.

In some aspects, the antibody or antigen-binding fragment thereof comprises a VL comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 6 or SEQ ID NO: 22.

In some aspects, the antibody or antigen-binding fragment thereof comprises a VH encoded by a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 3 or SEQ ID NO: 29.

In some aspects, the antibody or antigen-binding fragment thereof comprises a VL encoded by a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 4 or SEQ ID NO: 20.

In some aspects, the sequence variation defined herein occurs outside of the CDR sequences. In some aspects, the sequence identity is over the full length of the defined sequence (e.g., VH/VL).

In some aspects, the antibody or antigen-binding fragment thereof comprises a VH comprising the amino acid sequence of SEQ ID NO: 5 or SEQ ID NO: 21 (i.e., 100% identical).

In some aspects, the antibody or antigen-binding fragment thereof comprises a VL comprising the amino acid sequence of SEQ ID NO: 6 or SEQ ID NO: 22 (i.e., 100% identical).

In some aspects, the antibody or antigen-binding fragment thereof comprises a VH/VL pairing as set forth in SEQ ID NO: 5/SEQ ID NO: 6 or SEQ ID NO: 21/SEQ ID NO: 22.

In some aspects, the antibody or antigen-binding fragment thereof defined herein is from a human, primate, non-human primate (e.g., monkey), mouse, rat, rabbit, hamster, donkey, sheep, or cow. The antibody or or antigen-binding fragment thereof may be or derived from an IgG, IgD, IgE, IgM, or IgA isotype. The IgG may be IgG1, IgG2, IgG2a/2c, IgG2b, IgG3, or IgG4 subclass.

In some aspects, the antibody or antigen-binding fragment thereof defined herein may be a scFv, scFv-Fc, minibody, monovalent IgG, diabody, triabody, tetrabody, Fab, Fab′, Fv, F(ab′)2, or any combination thereof. In some aspects, the antibody or antigen-binding fragment thereof defined herein may be a kappa-lambda body, a dual-affinity re-targeting molecule (DART), a knob-in-hole, a strand-exchange engineered domain body (SEEDbody), a Bispecific T cell engager (BiTe), a CrossMab, an Fcab, a Diabody, a Tandem diabody (TandAb), or a DuoBody. In some aspects, the antibody or antigen-binding fragment thereof defined herein is humanized.

In some aspects, the antibody or antigen-binding fragment thereof defined herein specifically binds to full-length native alpha-synuclein. In some aspects, the antibody or antigen-binding fragment binds to specific epitopes or fragments of aSyn (e.g., aSyn fragment peptide of SEQ ID NO: 2). In some aspects, the aSyn is a human aSyn In some aspects, the human aSyn comprises the amino acid sequence of SEQ ID NO: 1, or any variant, isoforms, homologs, analogs, orthologs, or paralogs thereof. In some aspects, the antibody or antigen-binding fragment thereof defined herein may specifically bind to a polypeptide that is at least 50%, 55%, 60%, 65%, 70%, 75%, 80%, 85%, 90%, 95%, or 99% identical to SEQ ID NO: 1, or any fragment thereof.

In some aspects, the antibody or antigen-binding fragment thereof defined herein specifically binds to modified full-length aSyn or fragments thereof. Modifications in aSyn may include but are not limited to mutants (e.g., addition, substitution, or deletion mutations in SEQ ID NO: 1) or post-translational modifications (e.g., chemical or post-translational modifications such as acetylation, carboxymethylation, deamidation, phosphorylation, citrullination, homocitrullination, glycosylation, oxidation, sulfatation, sumoylation, prenylation, ubiquitination, etc.).

In some aspects, the antibody or antigen-binding fragment thereof defined herein specifically binds to phosphorylated alpha-synuclein. aSyn may be phosphorylated at any residue. In some aspects, aSyn is phosphorylated at a serine residue. In some aspects, aSyn is phosphorylated at residue serine 129. In some aspects, serine 129 corresponds to serine 129 of SEQ ID NO: 1 (or serine 19 of SEQ ID NO: 2) or at a residue corresponding to, positionally-equivalent to, or homologous to serine 129 of SEQ ID NO: 1 (or serine 19 of SEQ ID NO: 2). In some aspects, the aSyn or phosphorylated alpha-synuclein is from mouse or human.

In some aspects, the antibody or antigen-binding fragment thereof defined herein reduces the levels or expression of phosphorylated alpha-synuclein when administered in vivo. In some aspects, the antibody or antigen-binding fragment thereof defined herein does not affect the levels or expression of native unphosphorylated alpha-synuclein when administered in vivo.

In some aspects, there is described a polynucleotide encoding the antibody or antigen-binding fragment thereof defined herein.

In some aspects, there is described a vector comprising the polynucleotide encoding the antibody or antigen-binding fragment thereof defined herein. The vector may be a retroviral vector, a DNA vector, a plasmid, an RNA vector, an adenoviral vector, an adenovirus associated vector, a lentiviral vector, or any combination thereof.

In some aspects, there is described a cell comprising the polynucleotide or the vector defined herein. The cell may be any cell (e.g., mammalian cell), such as an immune cell. The cell may further be a chimeric antigen receptor (CAR) immune cell (e.g., CAR T cell or CAR NK cell).

In some aspects, there is described a chimeric antigen receptor (CAR) comprising the antibody or antigen-binding fragment thereof defined herein.

In some aspects, there is described a polypeptide comprising the antibody or antigen-binding fragment thereof defined herein.

In some aspects, there is described an in vitro method of making a cell expressing the antibody or antigen-binding fragment thereof defined herein comprising transducing the cell with the polynucleotide defined herein under suitable conditions. In some aspects, there is described an in vitro method of making the antibody or antigen-binding fragment thereof defined herein comprising incubating the cell defined herein under suitable conditions. Suitable conditions for cell transduction and protein (e.g., antibody) expression/production/secretion/purification are known in the art. In some aspects, the method further comprises isolating the cell.

In some aspects, there is described a pharmaceutical composition comprising the antibody or antigen-binding fragment thereof defined herein, the vector defined herein, the cell or cells defined herein, the CAR defined herein, or the polypeptide defined herein, and a pharmaceutically acceptable carrier, excipient, diluent, solubilizer, emulsifier, preservative and/or adjuvant.

In some aspects, there is described a use of the antibody or antigen-binding fragment thereof defined herein, the vector defined herein, the cell or cells defined herein, the CAR defined herein, the polypeptide defined herein, or the pharmaceutical composition defined herein for the treatment of a synucleinopathy, the detection, diagnosis, or prognosis of a synucleinopathy, or the monitoring of the progression of a synucleinopathy in a subject.

In some aspects, there is described a use of the antibody or antigen-binding fragment thereof defined herein, the vector defined herein, the cell or cells defined herein, the CAR defined herein, the polypeptide defined herein, or the pharmaceutical composition defined herein for the manufacture of a medicament for the treatment of a synucleinopathy in a subject.

In some aspects, there is described a method for treating a synucleinopathy in subject in need thereof, said method comprising administering the antibody or antigen-binding fragment thereof defined herein, the vector defined herein, the cell or cells defined herein, the CAR defined herein, the polypeptide defined herein, or the pharmaceutical composition defined herein in the subject.

In some aspects, the antibody or antigen-binding fragment thereof defined herein, the vector defined herein, the cell or cells defined herein, the CAR defined herein, the polypeptide defined herein, or the pharmaceutical composition defined herein can be used at any dose and be administered via any route. Dosages may include any therapeutically-effective dosages. The antibody or antigen-binding fragment thereof defined herein, the vector defined herein, the cell or cells defined herein, the CAR defined herein, the polypeptide defined herein, or the pharmaceutical composition defined herein may further be used in combination with any approved treatments or therapies (e.g., any standard of care therapy for a synucleinopathy) and in any administration sequence.

In another aspect, there is described a kit for use in diagnosing/prognosing or determining the progression of a synucleinopathy in a subject. Said kit may comprise the antibody or antigen-binding fragment thereof defined herein and one or more reagents for alpha-synuclein or phosphorylated forms thereof in a sample (e.g., biological sample from a subject). The kit may further include a plate (such as a microplate), tube, or any suitable support, upon which a protein (such as an aSyn) is or may be immobilized. Reagents for the kits described herein may include but are not limited to reagents commonly used in an immunoassay, such as in an enzyme-linked immunosorbent assay (ELISA). For example, the reagents may include a detecting antibody for the detection of the complex formed by aSyn and the antibody or antigen-binding fragment thereof defined herein. In some aspects, the kit may comprise instructions to perform the methods described herein.

In some aspects, there is described a method for the detection, diagnosis, or prognosis of a synucleinopathy, or for the monitoring of the progression of a synucleinopathy, in a subject, said method comprising detecting alpha-synuclein (aSyn) in the subject or a biological sample from said subject with the antibody or antigen-binding fragment thereof defined herein. The method may further comprise comparing the levels of aSyn or phosphorylated aSyn (e.g., as described above) to a reference or control subject or a sample from said reference or control subject. In some aspects, the method further comprises administering the antibody or antigen-binding fragment thereof in said subject.

In some aspects, there is described an in vitro or ex vivo method of detecting alpha-synuclein (aSyn) in a sample (e.g., biological sample), said method comprising contacting the sample with the antibody or antigen-binding fragment thereof defined herein to form a complex between the antibody or antigen-binding fragment thereof and alpha-synuclein; detecting the complex between the antibody or antigen-binding fragment thereof and alpha-synuclein; and optionally, measuring the levels of the complex between the antibody or antigen-binding fragment thereof and alpha-synuclein.

In some aspects, the sample defined herein is a biological sample from a human subject having or suspected of a synucleinopathy.

In some aspects, the methods described herein are used to detect or quantify the levels phosphorylated aSyn (e.g., aSyn phosphorylated at serine 129 of SEQ ID NO: 1 or at a residue corresponding to, positionally-equivalent to, or homologous to serine 129 of SEQ ID NO: 1).

In some aspects, the synucleinopathy described herein is Parkinson's disease (PD), Lewy body disease (LBD), dementia with Lewy bodies, Multiple systemic atrophy (MSA), pure autonomic failure, or rapid eye movement (REM) sleep behaviour disorder.

In some aspect, the antibodies or antigen-binding fragments defined herein have a higher binding affinity (e.g., lower K_{D} value), avidity, and/or specificity to either unphosphorylated (e.g., WT) or phosphorylated aSyn (e.g., serine 129), than known or conventional aSyn antibodies (e.g., commercially available antibodies). In some aspects, the antibodies or antigen-binding fragments defined herein are more stable (e.g., in vivo) than known or conventional aSyn antibodies (e.g., commercially available antibodies). In some aspects, the antibodies or antigen-binding fragments defined herein are functionally superior to known or conventional aSyn antibodies (e.g., commercially available antibodies). For example, treatment/prevention of synucleinopathies or associated symptoms, prevention of aSyn aggregates (e.g., Lewy bodies in the brain), and/or prevention of alpha-synuclein fibrils internalization in neurons. In some aspects, the antibodies or antigen-binding fragments defined herein are effectively internalized by brain cells (e.g., neurons, microglia) and bind intracellular alpha-synuclein aggregates (e.g., Lewy Bodies). In some aspects, the antibodies or antigen-binding fragments defined herein have an enhanced ability to become internalized by brain cells containing alpha-synuclein aggregates as compared to known or conventional aSyn antibodies (e.g., commercially available antibodies). In some aspects, the antibodies or antigen-binding fragments defined herein have an enhanced ability to bind intracellular, extracellular, or both intracellular and extracellular alpha-synuclein aggregates as compared to known or conventional aSyn antibodies (e.g., commercially available antibodies).

Other objects, advantages and features of the present description will become more apparent upon reading of the following non-restrictive description of specific embodiments thereof, given by way of example only with reference to the accompanying drawings.

### EXAMPLES

### Example 1: Materials and Methods

### Cell Culture

HEK cells were maintained in DMEM (cat #11995065) supplemented with 10% ultralow IgG fetal bovine serum (Gibco™ Canada 16250078cat) and 1% penicillin-streptomycin (cat #SV30010). For immunocytochemistry, cells were plated on Poly-L-lysine (cat #P4707)-coated glass coverslips (cat #12-545-81) at a density of 50K/well. The following day, cells were transfected with 500 ng DNA/well using Lipofectamine™2000 and incubated for 48 h at 37 °C. Cells were fixed in 4% PFA on ice for 20 min and washed with PBS afterwards. For western blots experiments, cells were plated in 6-well plates at a density of 400K cells/well and transfected the next day with 3 μg total DNA/well using Lipofectamine2000. 48 h later, cells were lysed with RIPA buffer (NEB #9806S) supplemented with PMSF on ice for 5 minutes. Lysates were transferred to 1.5 mL tubes, sonicated briefly, and centrifuged at 14000 g for 20 minutes at 4 C. Supernatant was transferred to a clean tube and total protein concentration was measured with the DC protein assay (Bio-Rad™ 500-0112). 4× Laemmli™ Sample buffer (Bio-Rad #1610747) was added at the appropriate concentration and the lysates were boiled 5 min at 95 °C. Final lysates were aliquoted in working volumes and stored at −80 °C.

### Monoclonal Antibody Generation and Testing

Six mice were injected with a 26AA peptide corresponding to the C-terminal part of aSyn (SEQ ID NO: 1) with a phosphorylated serine 129 (serine 129 in SEQ ID NO: 1 or serine 19 in SEQ ID NO: 2), coupled to KLH (Keyhole Limpet Hemocyanin). The mice whose blood contained antibodies against the peptide (verified by ELISA) were immunized again twice. Splenocytes of these mice were extracted and fused with myelocytes to produce hybridomas. Splenocytes were selected by passaging through various media and diluted to produce clones (Mitra & Tomar, 2021). Clones were screened by ELISA using the peptide conjugated to BSA (bovine serum albumin) to verify which ones were binding to the injected peptide.

Positive clones were tested by Western blot and immunocytochemistry (ICC) to verify specificity against phosphorylated versus total aSyn protein. For ICC, HEK cells were plated on coverslips and transfected with either Human aSyn-WT, Human aSyn-WT+PLK2, Human aSyn-S129G+PLK2, or GFP. After fixation, cells were incubated with a blocking buffer (1×PBS+5% normal donkey serum+1.5% TritonX™-100) for 1 h at RT. Hybridoma supernatants were used as primary antibodies O/N at 4 C. The next day, cells were washed with PBS, and secondary antibody (AlexaFluor™647 anti-mouse) was added in blocking buffer for 1 h at RT. Cells were mounted with DAPI-containing mounting media (Cat #P36935). For Western blot, 20 μg DNA was loaded in a pre-casted SDS-PAGE gel (Bio-Rad #4568093) and the gel was run for 90 min at 100V. Proteins were transferred on a nitrocellulose membrane using the TransBlot™ Turbo™ apparatus (Bio-Rad). Membranes were blocked for 1 h in TBST containing 5% skim milk and then incubated O/N at 4 C in the hybridoma supernatants. The next day, membranes were washed in TBST and incubated 1 h at RT in 5% milk with the secondary antibody (HRP-anti-mouse).

### Monoclonal Antibody Purification

Monoclonal antibodies were purified from hybridoma supernatant. Briefly, Protein-G-Agarose resin was added to a 50 mL tube and washed 2 times with TBS. Hybridoma supernatant was added to the tube and incubated overnight with slow agitation at 4 °C. The next day, the tube was centrifuged, and the supernatant was discarded. The resin was then washed 3 times with TBS, and a glycine solution (pH 3.5) was added to detach the antibody from the resin. After a 5 minutes incubation, the tube was centrifuged, and the supernatant was collected and placed in another tube containing 1M Tris. The purified antibody was then concentrated with a Vivaspin™ 6 column with a 10 kDa molecular weight cut off membrane. Concentration was read with a Nanodrop™ system.

scFv Synthesis

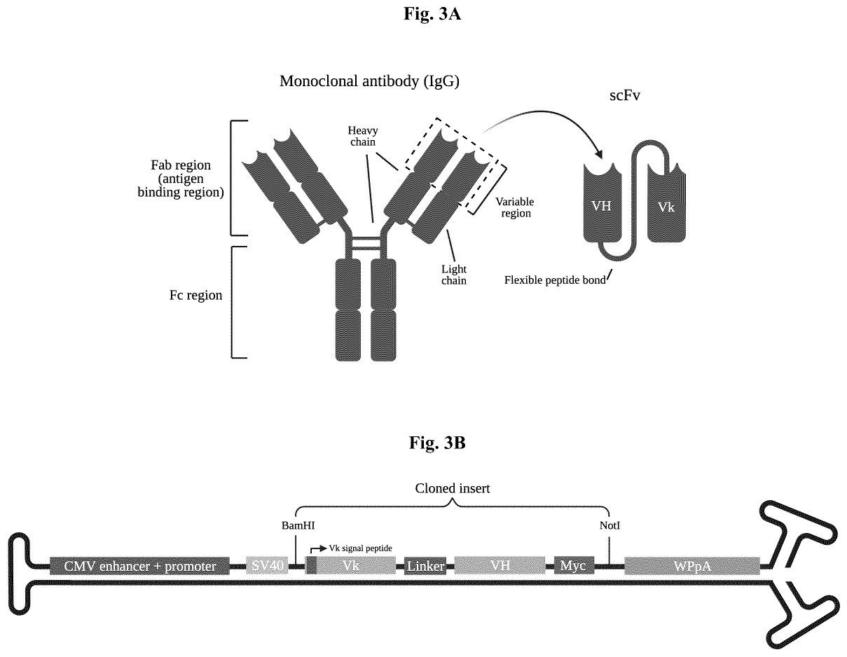

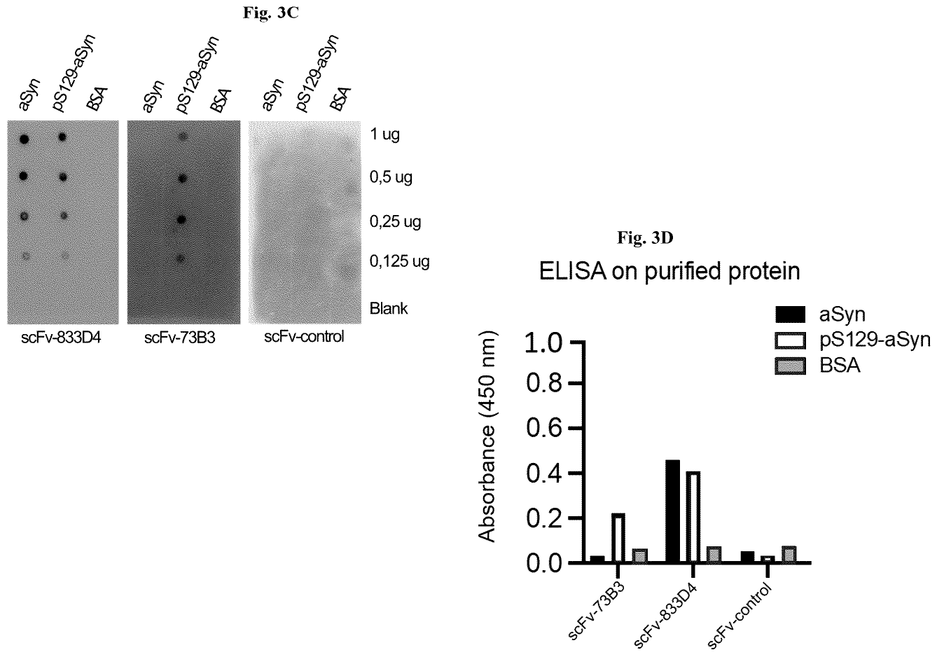

The variable regions (VL and VH) of the selected monoclonal antibodies were sequenced and the DNA sequences encoding those regions were used to design in silico scFv open reading frames in either Vk (including the sequence encoding the leader peptide)-linker-VH (without leader peptide)-HA tag-human c-Myc-epitope direction (5′ to 3′ direction), or VH (including the sequence encoding the leader peptide)-linker-Vk (without leader peptide)-HA tag-human c-Myc-epitope direction (5′ to 3′ direction), framed by restriction sites for recombinant restriction enzymes BamHI (5′) and NotI (3′). Double-stranded DNA encoding these ORFs were synthetized by primer extension (by Bio-Basic™ Canada) and cloned into plasmid Bluescript-SK-II™ (Stratagene™ Cat. No 212205). Those sequences were inserted in an expression plasmid for scAAV (FIG. 3B). Briefly, the sequences were cloned in an pGEM 4Z-derived expression plasmid (Promega™, Cat P2161) between the ITRs of scAAV-2 using standard recombinant DNA techniques. Plasmids were transformed in competent Stable (New England Biolabs, Cat C3040H) bacteria and were plated on amp-containing agar in petri dishes. Colonies were picked and let O/N to grow. DNA was extracted using a commercial kit (Qiagen™ Cat. No./ID: 27104). Construct sequences were verified by digestion with restriction enzymes and by sequencing. Sequences for expression of the antibody-derived protein contain a CMV promoter/enhancer, an in-frame Myc tag in C-terminal, and a WPRE and SV40 polyadenylation signal for enhanced expression.

### Dot Blots

aSyn monomers (Proteos™ #RP-003), pS129-aSyn monomers (Proteos #RP-004) and BSA (Sigma #A9647) were manually blotted on nitrocellulose membranes. Each dots contains 2 μg of total protein in 2 ul volume. aSyn and pS129-aSyn were diluted with BSA solution to achieve constant protein amount. Membranes were let to dry for 15 min a RT and were then humidified with 1×TBS before being stained with ponceau red. Images of ponceau staining were obtained with Bio-Rad's Chemidoc. After destaining with water, membranes were blocked with TBST containing 5% skim milk for 1 h at RT. HEK cells medium containing the secreted scFv was used as a primary antibody for O/N at 4 C. The next day, membranes were incubated with an HRP-conjugated myc antibody (ab62928) at a dilution of 1:10 000 for 1 h at room temperature. Images were acquired with Bio-Rad's Chemidoc™ using ECL substrates (Cat #1705062).

### ELISA

ELISA high binding plates were coated by incubating with a solution of purified alpha-synuclein (20 ug/mL) in carbonate binding buffer overnight at 4 °C. The next day, the plate was washed 3 times with wash buffer (1×PBS containing 0.05% Tween™ 20) and then incubated for 1 h at room temperature in blocking buffer (3% fish gelatin in PBS). After blocking, the plate was washed, and primary antibody diluted in 1% fish gelatin in PBS or filtered scFv-containing cell media was added and incubated for 2 h at room temperature. The plate was washed again and incubated for 30 min at 37C with secondary antibody conjugated to HRP (see supplementary material). After washing, 100 μl of Tetramethylbenzidine solution (Sigma™ T4444) was added and incubated at 37C for 5-10 minutes. 100 μl of 1M HCl was added to each well to stop the reaction and the absorbance at 450 nm was read with the Victor™ Nivo™ plate reader (PerkinElmer™). Experiment was repeated two individual times.

### Mice

A53T α-synuclein transgenic line M83 (B6;C3-Tg(Prnp-SNCA*A53T)83Vle/J, JAX stock #004479) was used. Mice were breed in house by crossing homozygous females with hemizygous males. Pups were genotyped using ddPCR according to Jackson laboratory's protocol. Hemizygous males and females aged between 60 and 90 days were used. Mice were housed in a light-dark standard cycle with access to water and food ad libitum and EnviroDry™ was added to their cage to encourage nesting. Males were housed separately because aggressive behaviour was frequently observed between housemates. All mice were weighted weekly starting at 8 weeks post-PFF injection.

### In Vivo Injections

Mice were injected with aSyn pre-formed fibrils (PFF) in the right dorsal striatum (coordinates: A/P+0.20; M/L+2.00; D/V 2.6) under isoflurane anesthesia. The needle was left in place for 5 minutes after injection, and then slowly pulled back. Before injections, PFF were sonicated in a Bioruptor™ Pico bath for 20 min (30 sec. ON, 30 sec. OFF, 20 cycles) at 10 °C PFF were injected less than 4 h after sonication to avoid degradation. A total of 5 ug of PFF in a volume of 2 μl was injected.

7 days after PFF injection, mice were injected intravenously in the retro-orbital sinus with the AAVB10-scFvs. Briefly, mice were anesthetized with isoflurane and a solution of alcaïne was applied on each eye for 30 sec. Insulin syringes were used to inject 100 ul of AAV diluted in Ringer's lactate. 5E11 GCs of virus was injected per mouse.

### Behavioral Tests

### Clinical Scoring

Starting 8 weeks post-injection, mice were monitored for motor impairments using the grid presented in supplementary. Three aspects were evaluated: clasping, gait, and ledge walking. Each test can be scored from 0 to 3 for a maximum of 9 points for all tests.

### Rotarod

Mice were placed in the behavior room 1 h prior to the test for habituation. For 2 days before the test. Mice were trained by placing them on the rod (company) for 5 min at 4 RPM constant speed. The third day, mice were tested in 3 trials of 5 min with speed increasing from 4 to 40 RPM. 5 minutes breaks were allowed between trials where the mice remained in the apparatus.

### Cylinder Test

Mice were placed in the behavior room for 1 h prior to the test for habituation. Mice were place in a glass cylinder with bottom covered in litter and were filmed from above for 6 minutes. The first minute was excluded from analysis. The number and direction of the turns and the forepaw touches to the wall were analyzed.

### Grip Strength Test

Mice were placed in the behavior room for 1 h prior to the test for habituation. The grip strength test from Biosed company was used (#EB1-BIO-GS4). Mice were held by the tail and were lowered on the grid (#EB1-BIO-GRIPGS) until their 4 paws gripped and were then pulled with constant strength horizontally until the hindlimbs let go. Maximum strength displayed was noted and 6 trials were made. Maximal and minimal values were excluded from analysis and a mean of the 4 remaining values was normalized on the mice weight.

### Tissue Processing

For histopathological analysis, mice were anesthetized with ketamine-xylazine and were sacrificed by cardiac perfusion with cold PBS followed with 4% paraformaldehyde. Brains and spinal cords were extracted and post-fixed for 90 min in 4% PFA, then transferred in 30% sucrose solution for O/N at 4 C. Brains and spinal cords were frozen in OCT and cut coronally at 60 μm with a cryostat (Leica #). Ipsilateral cortex was marked with a blade to recognized injected side. For western blot analysis, mice were sacrificed by cervical dislocation without anesthesia. Brains were extracted and cortex, hippocampus, midbrain, striatum, and medulla were dissected and frozen quickly in liquid nitrogen. The tissue was stored at −80C until used. Upon thawing on ice, tissue was lysed manually with a potter in RIPA buffer (NEB #9806S) supplemented with PMSF (NEB #8553S). After quick sonication, lysates were centrifuged at 14000 g for 20 min at 4 C. Supernatant was transferred to a clean tube and total protein concentration was measured with the DC protein assay II (Bio-Rad #500-0112). Samples were aliquoted and stored at −80 °C until used.

### Western Blotting

4× Laemmli sample buffer (Bio-Rad #1610747) was added, and samples were boiled for 5 min at 95 °C and then loaded on 4-20% Mini-PROTEAN® TGX Stain-Free™ Protein Gels (Bio-Rad #4568093). Gel was run at 100V for 90 minutes and proteins were transferred to a nitrocellulose membrane using the Bio-Rad Transblot Turbo system. For pS129-aSyn staining, membranes were fixed for 15 min in 4% PFA and then washed 3 times 10 min with 1×TBS. 5% milk in TBST was used as a blocking solution for 1 h at room temperature. Membranes were incubated with primary antibodies overnight at 4 °C (pS129-aSyn: Rabbit Abcam #ab51253, total aSyn: Rabbit CST2628, Actin: Mouse Millipore™ MAB1501)

### Immunohistofluorescence

Antigen retrieval was used for NeuN staining only. Briefly, brain sections were incubated in pre-heated sodium citrate buffer at 95-100C for 15 min and then washed 2 times in PBS with 0.05% TritonX-100. Sections were washed in 1×PBS and then incubated in blocking solution (1×PBS+5% NDS+0.3% TritonX-100 or 1×PBS+5% BSA+0.3% TritonX-100) for 1 h at RT. Sections were incubated O/N with primary antibodies in blocking solution (TH P60101 Pel-Freez™ Biological; NeuN MAB377; DAT MAB369; pS129-aSyn clone 73B3). The next day, sections were incubated with secondary antibodies (AlexaFluor 555, AlexaFluor 647, AlexaFluor 488) in blocking solution for 1 h at room temperature. If Amytracker (Amytracker 520 Ebba Biotech) was used, it was incubated for 30 minutes at a dilution of 1:1000 in PBS before mounting. Images for paSyn quantification were acquired with a slide scanner (Huron Technologies, #). All other images were acquired with the Zeiss™ LSM-710 confocal microscope and analyzed with ImageJ™ and Matlab™.

### Myc Staining

Staining of myc in brain and spinal cord sections was done with the Alexa Fluor 555 Tyramide Superboost™ kit (Invitrogen™ #B40923) according to the manufacturer's instructions.

### Cell Counting

For TH and NeuN cell counting, a stereological counting software was used (StereoInvestigator™ Offline, MBF Bioscience). 4 sections spaced of 3 wells were counted per animal. A surface of 55% of the SNc was counted manually in random places for the software to estimate total cell number.

### Phosphorylated Alpha-Synuclein and Amytracker™ Quantification

pS129-aSyn and Amytracker were quantified in 4 brain regions (thalamus, midbrain, medulla and hypothalamus) by counting the number of particles per area with the Fiji particle count plugin. 2 consecutive brain sections were analyzed per mouse.

### Statistical Analysis

All statistical analyzes were performed using GraphPad™ 9.4.0. Statistical significance was determined by unpaired two-tailed Student's t test for 2 groups comparisons, or one-way ANOVA test with Tukey's correction for multiple groups comparisons. P values lower than 0.05 were considered significant. *P<0.05, **P<0.01, ***P<0.001, ****P<0.0001. Experimenters were not blind to the groups when performing analyses, except mentioned otherwise in the figure legend.

### Example 2: Results

### Monoclonal Antibody Generation and Characterization

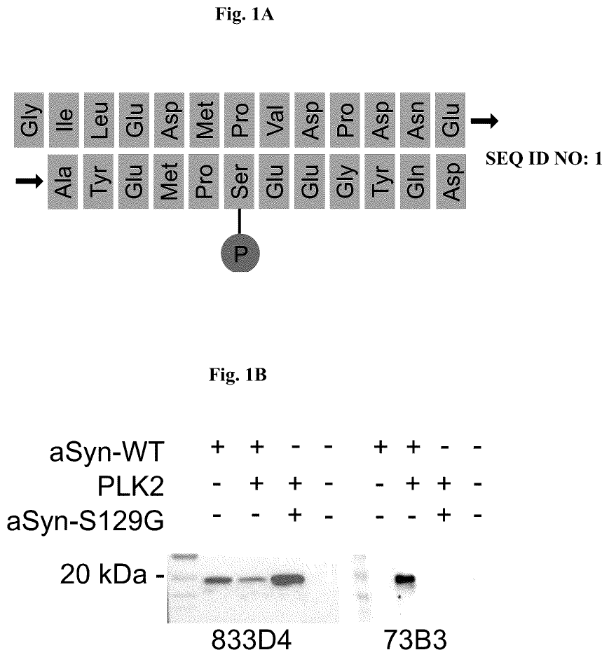

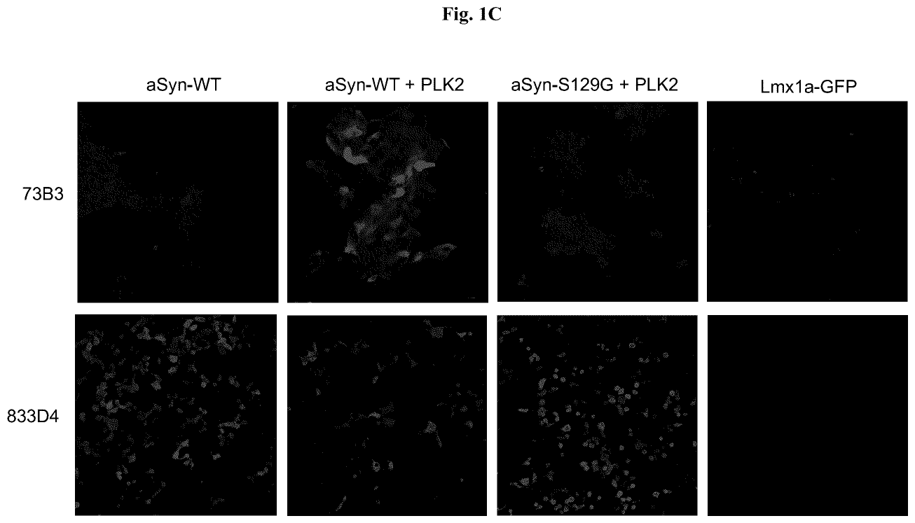

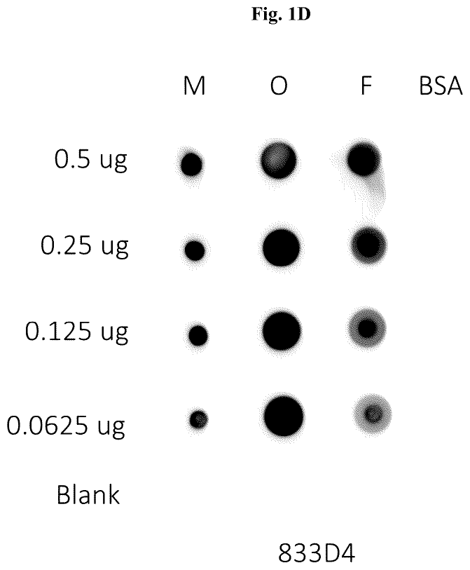

To produce anti-aSyn antibodies, mice were immunized 3× with a 25 amino-acid peptide (SEQ ID NO: 1) corresponding to a region near the C-terminal part of aSyn with a phosphorylated serine 129 (serine 19 of SEQ ID NO: 1) (FIG. 1A). The peptide was coupled to KLH. Splenocytes were harvested from three (3) mice that developed the strongest positive immune responses against the peptide and, were fused to NSO myeloma cells to generate hybridomas. Hybridoma were plated at 10, 1 and 0.1 cell per well, and supernatants from clonal hybridomas tested by ELISA against the peptide crosslinked to BSA. Hybridoma supernatants from ELISA-positive clones was used as primary antibody solutions in western blot experiments on HEK cells lysates. We selected an antibody that showed specificity to pS129-aSyn (73B3) and an antibody that showed specificity to all forms of aSyn tested (833D4) (FIG. 1B). To ensure that the antibodies could bind undenatured aSyn, we also applied the hybridoma supernatants as primary antibody in immunocytofluorescence on transfected HEK cells. Both antibodies demonstrated the same specificity as observed in western blot (FIG. 1C). To further characterize the monoclonal antibodies, we performed dot blots by blotting monomers, oligomers, and fibrils of aSyn and incubating with our total aSyn-specific antibody (833D4). Affinity data for 833D4 is shown in FIG. 1D.

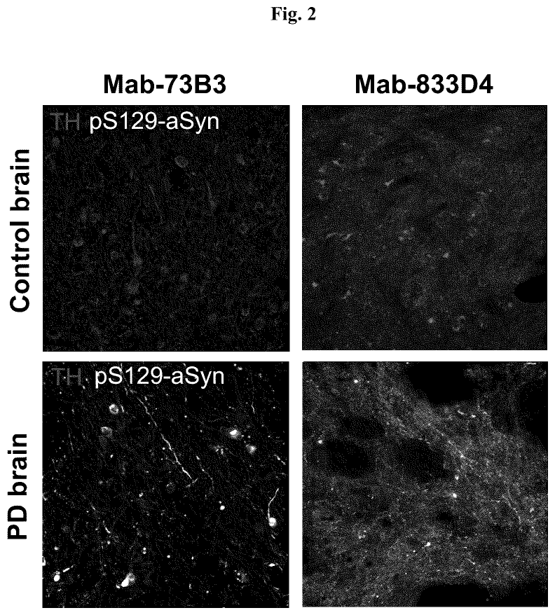

The two IgGs were then purified from the hybridoma culture media using Protein G agarose beads and were tested on human PD (Parkinson's disease) and control brain sections to detect pathological aSyn. The phospho-specific antibody (73B3) recognized only aSyn in the PD brains and not in the control aged-matched brains. Detected structures resemble those of Lewy neurites and Lewy bodies (FIG. 2). The total aSyn-specific antibody (833D4) also recognized Lewy-like pathology in PD brains, though they also showed some basal staining in control brains. This is consistent with the fact that those antibodies also recognized non-phosphorylated aSyn.

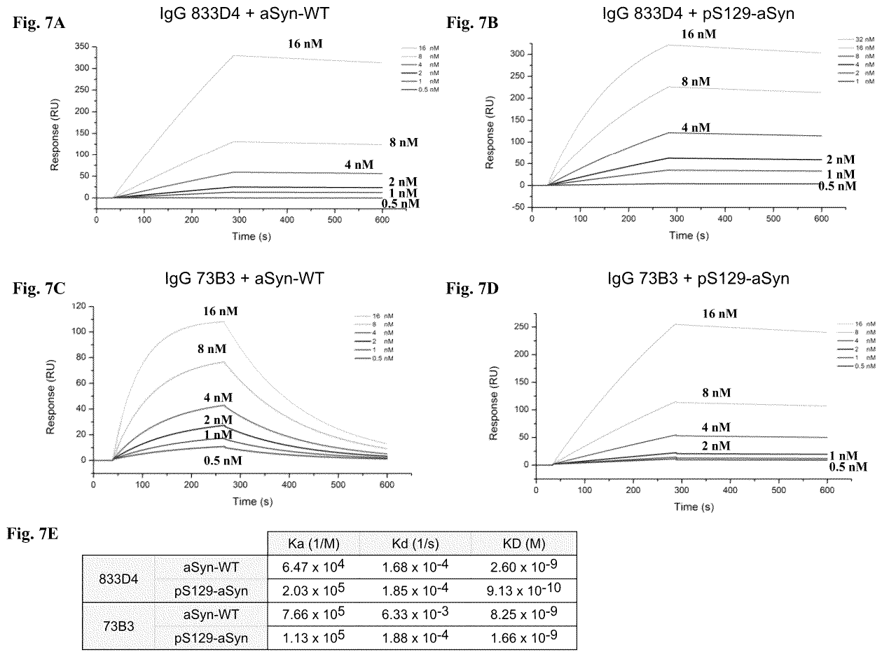

Binding and specificity to aSyn-WT and pS129-aSyn was confirmed by surface plasmon resonance (SPR) (FIGS. 7A-7E). As shown, Mab 833D4 was shown to bind to both aSyn-WT and pS129-aSyn with high specificities. Mab 73B3 was, however, shown to have higher binding specificity for pS129-aSyn as compared to aSyn-WT. With respect to aSyn-WT, Mab 73B3 binding along was observed but with rapid dissociation by SPR, which explains the lack of binding to aSyn-WT by immunoassays (FIGS. 1B, 1C, and 3D). Binding to pS129-aSyn by Mab 73B3, however, was maintained and no rapid dissociation was observed. These findings are reflected by the Ka (association), Kd (dissociation), and KD (equilibrium) values (FIG. 7E).

These data suggest that potent antibodies specific to aSyn and pS129-aSyn or selectively specific to pS129-aSyn were successfully generated. These data further suggest that Mab 73B3 may have a higher binding affinity pS129-aSyn as compared to aSyn-WT, and thus demonstrating greater specificity to pS129-aSyn, whereas 833D4 binds to both aSyn-WT and pS129-aSyn with high binding affinities.

scFv Generation and Validation

scFvs were generated by cloning the variable region of the light (Vk or VL) and the heavy chain (VH) of the monoclonal antibodies (FIG. 3A) in an expression plasmid containing a Cytomegalovirus (CMV) immediate/early promoter/enhancer, a Simian Virus 40 (SV40) small intron, and a Woodchuck Hepatitis Virus (WHV) Posttranscriptional Regulatory Element (WPRE) coupled to a SV40 polyadenylation signal sequence for increased expression. In the plasmid, this expression unit was framed on each side by Inverted Terminal Repeat (ITR) elements from the AAV-2 virus. The two variable regions of the scFv were spaced by an in-frame Gly(Ser)4 peptide bridge, and a Myc tag was added in frame at the end of the scFv coding region to facilitate detection (FIG. 3B). To verify that the scFvs retained their binding specificity, we transfected HEK cells with the expression plasmids encoding the scFvs and used the cell media as a primary antibody solution in dot blot and ELISA on purified aSyn protein. The scFv-73B3 kept its binding specificity for pS129-aSyn. The scFv-833D4 kept its binding specificity for both total aSyn and pS129-aSyn (FIG. 3C). These results were confirmed in ELISA on purified aSyn and pS129-aSyn proteins (FIG. 3D). As control we used commercially available antibodies against total aSyn and pS129-aSyn (CST-2628 and Wako).

These data suggest that potent antibody fragments specific to aSyn and pS129-aSyn or selectively specific to pS129-aSyn were successfully generated.

Systemically Administered scFv-mAbs Efficiently Protected Against Motor Impairment and aSyn Pathology in an Aggressive Synucleinopathy Mouse Model

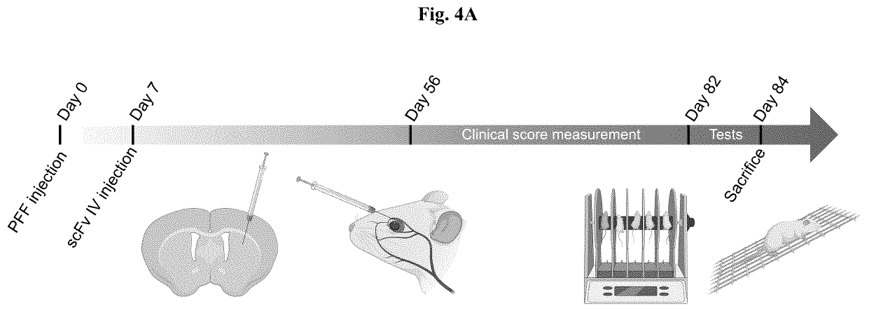

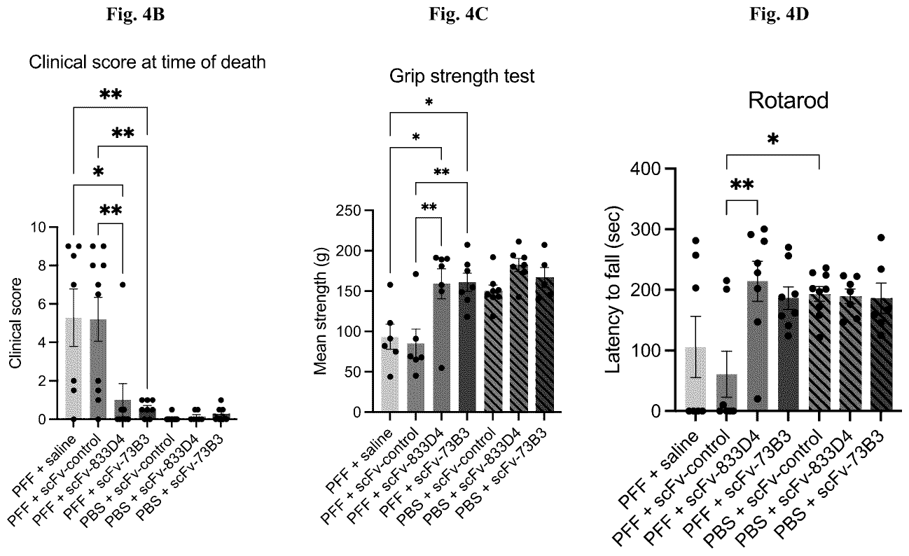

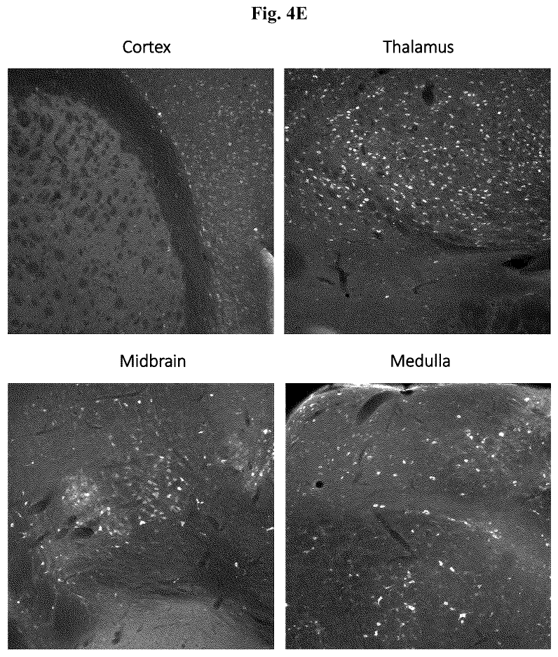

The plasmids encoding the anti-aSyn scFvs were co-transfected with a plasmid encoding an expression cassette for the AAVCap-B10 viral capsid, and a plasmid encoding other AAV vector and were administered intravenously in the retro-orbital sinus 7 days after PFF injection (FIG. 4A). Starting 8 weeks later, mice were closely monitored with a scoring system to detect any motor impairment. scFv-833D4 and 73B3 significantly protected against paralysis, as observed with the significantly higher clinical score at the time of death for the mice that received the control scFv or no scFv, compared to the ones treated with scFv-833D4 and 73B3 (FIG. 4B). Moreover, mice treated with the scFv-833D4 and 73B3 were significantly stronger in the grip strength test compared to control-scFv and no scFv groups at 12 weeks post-PFF injection (FIG. 4C). Finally, mice treated with the scFv-833D4 and 73B3 were staying significantly longer on the accelerating rotarod compared to control-scFv and no scFv groups (FIG. 4D). Successful brain expression of the scFvs was observed using the monoclonal anti-Human Myc tag as primary antibody, and the Alexa Fluor 555 Tyramide Superboost™ kit (Invitrogen™ #B40923) according to the manufacturer's instructions (FIG. 4E). Amplification of the signal was necessary since scFvs are very small and do not accumulate in the cell cytoplasm but are instead secreted in the extracellular space.

12 weeks after PFF injection and without treatment, aggregates of paSyn are largely present in various brain regions, with emphasis on posterior regions. This is consistent with the expression pattern of the transgene in the M83 transgenic line. Treatment with scFv-833D4 and 73B3 significantly reduced paSyn pathology in hypothalamus, midbrain, PAG and medulla compared to control scFv and no scFv groups which suggest a protection against pathological spread of aSyn (FIGS. 5A-5D). At 12 weeks after injection, no significant loss of TH+ neurons in the SNc or TH+ axons in the striatum are observed in any group.

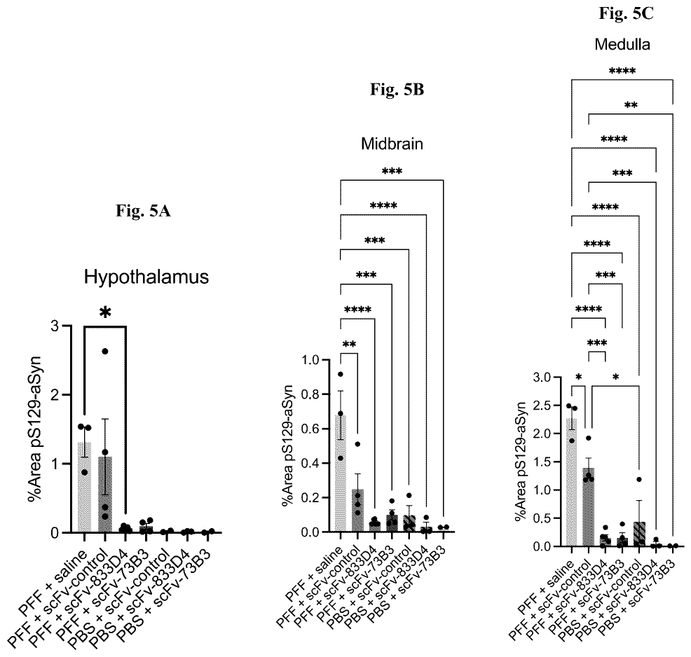

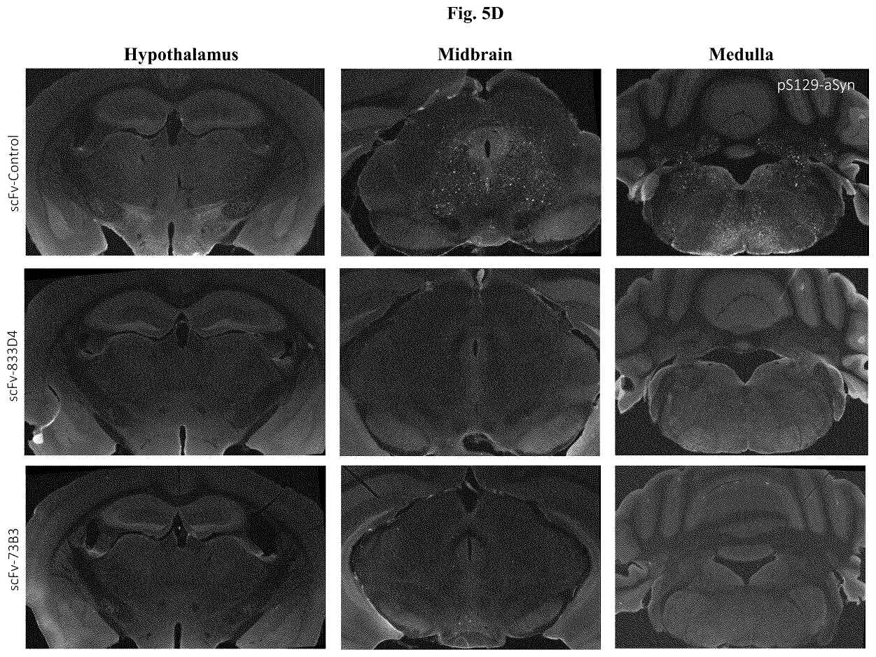

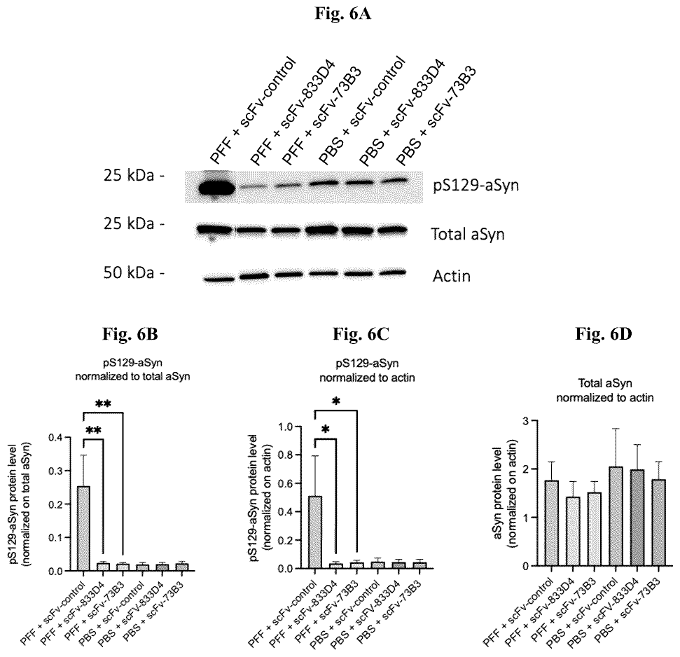

Furthermore, treatment with PFF and either scFv-73B3 or scFv-833D4 in mice significantly reduced pS129-aSyn levels in brain extracts (FIGS. 6A-6C). Interestingly, total aSyn levels (i.e., unphosphorylated aSyn) in the brain extracts of mice were unaffected by treatment with either scFv-73B3 or scFv-833D4 (FIGS. 6A and 6D). These data suggest that 73B3 and 833D4 mAbs do not affect native unphosphorylated aSyn levels in mice.

These data suggest that monoclonal antibodies specific to pS129-aSyn can be used as a potential and safe treatment for Parkinson's disease (PD) and other synucleinopathies. These data further suggest that 73B3 and 833D4 mAbs preferentially bind to pS129-aSyn relative to aSyn in vivo.

### Monoclonal Antibodies 833D4 and 73B3 Prevent Alpha-Synuclein Aggregation

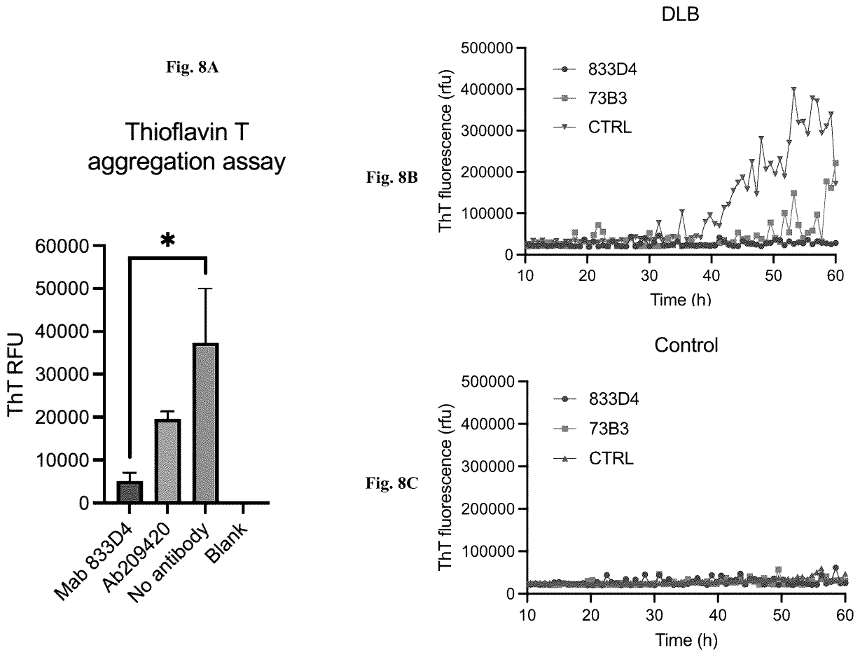

aSyn aggregation (e.g., Lewy Bodies) is the major neuropathological hallmark of several neurodegenerative diseases known as α-synucleinopathies, including Parkinson's disease (PD), dementia with Lewy bodies, and multiple system atrophy. Preventing and minimizing α-Syn aggregation and fibril formation is thus important in preventing and treating these diseases. FIGS. 8A-8C show the prevention of alpha-synuclein aggregation with antibody 833D4 in vitro. Thioflavin T (ThT) aggregation assay performed by incubating alpha-synuclein monomers at 37 °C with agitation for 5 days with or without addition of monoclonal antibody 833D4 or Abcam anti-aSyn antibody (Abcam ab209420; Clone MJFR1) in the reaction mix. As shown in FIG. 8A, 833D4 monoclonal antibodies were more effective in preventing alpha-synuclein aggregation as compared to the commercial antibody Abcam ab209420.

FIGS. 8B-8C further show the results of a Real-Time Quaking-Induced Conversion (RT-QuIC) assay where monoclonal antibody 833D4 are incubated with agitation in combination with Dementia with Lewy bodies (DLB) (FIG. 8B) or control (FIG. 8C) brain homogenates. Alpha-synuclein aggregation is measured with ThT. As shown in FIGS. 8B-8C, monoclonal antibody 833D4 was effective in preventing fibrilization as compared to controls.

FIG. 8D shows a comparison of phosphorylated aSyn (pS129) staining with clone 73B3 and a commercial anti-Phospho α-Synuclein Ser129 antibody from Wako (pSyn #64; FUJIFILM™ Wako Pure Chemical Corporation) in brain sections from PD mice and untreated mice. In the PD mouse model, the commercial Wako antibody shows a nuclear staining that does not resemble Lewy-like aggregates. This antibody also shows non-specific binding in control mice that should not exhibit pS129-aSyn pathology. Clone 73B3, however, was shown to have a clear and strong signal reminiscent of Lewy pathology (e.g., dystrophic neurites and cytoplasmic aggregates) in the PD mouse model, and doesn't show any binding in the control mice, and thus minimal non-specific binding.

FIG. 8E shows a comparison of phosphorylated aSyn (pS129) staining with clone 73B3 and a commercial anti-alpha-synuclein (phospho S129) antibody from Abcam (ab51253) in primary cultures of midbrain neurons with or without addition of aSyn preformed fibrils (PFF). The clone 73B3 (left side) detects aSyn inclusions in neurites (arrows) and cell body (arrow in box) in the condition with PFF, while the signal is almost absent in the absence of PFF. The commercial antibody ab51253 from Abcam (right side), however, shows strong signal in cell bodies (arrows) in the absence of any PFF treatment.

These data suggest a significant functional advantage of the antibodies defined herein over these known and commercially available anti-aSyn and anti-pS129-aSyn antibodies.

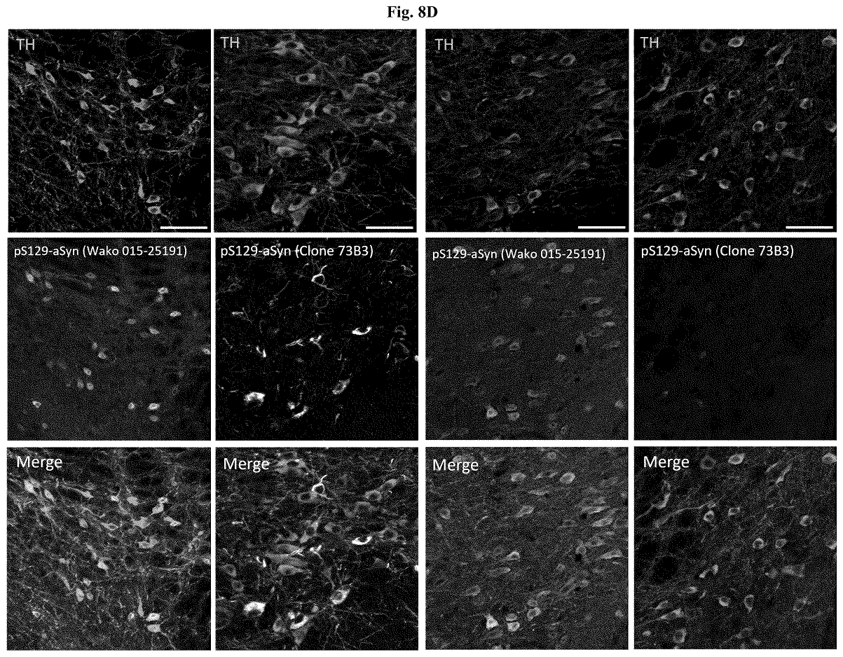

FIGS. 9A-9B show the prevention of alpha-synuclein fibril internalization in human neurons by 833D4 and 73B3 scFvs. Neurons were generated from human induced pluripotent stem cells (iPSCs). The neurons were infected with AAV-scFv 833D4 or 73B3 and then exposed for 2 h to alpha-synuclein preformed fibrils (PFF) tagged with AlexaFluor™ 488. As shown in in FIGS. 9A-9B, both 833D4 and 73B3 scFvs were effective in preventing internalization of alpha-synuclein fibrils in human neurons compared to control scFvs.

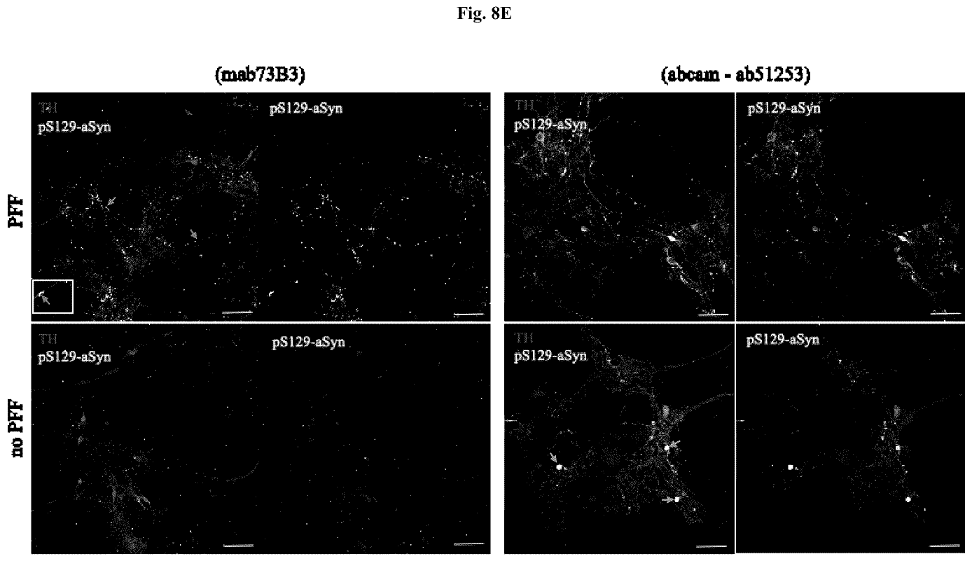

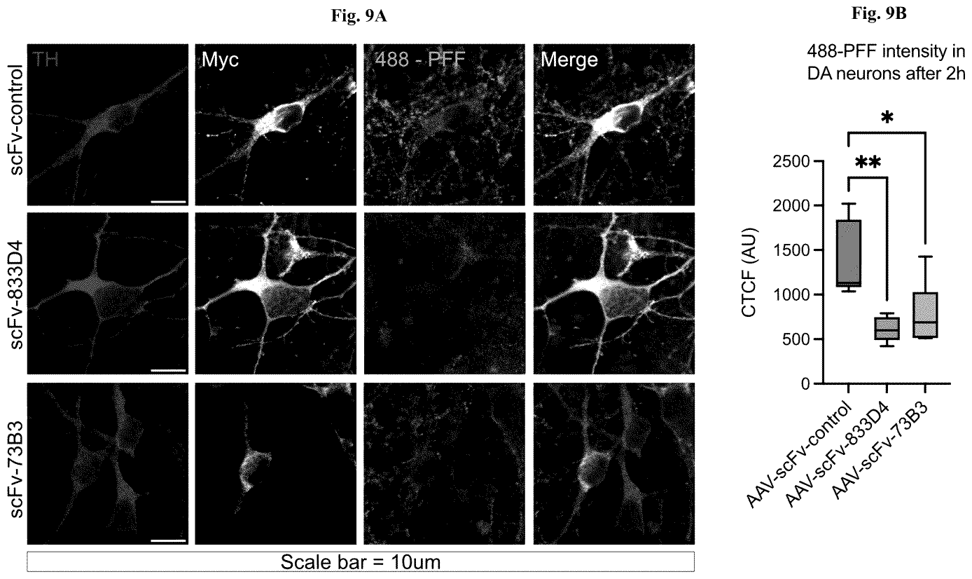

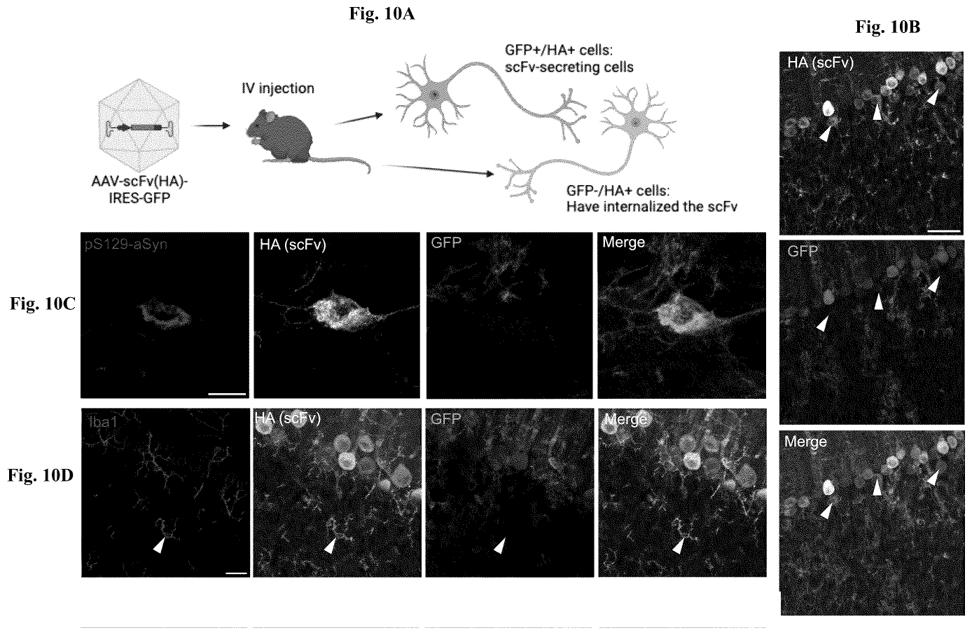

FIGS. 10A-10D show the secretion and internalization 833D4-scFvs by neurons and microglia. FIG. 10A shows a schematic of the experiment for the expression of scFv-secreting and non-secreting cells. Cells that are both HA and GFP positive have been infected by the vector and are secreting cells. Cells that are HA positive, but GFP negative were not infected by the vector and have internalized the 833D4-scFv. FIG. 10B shows fluorescence microscopy images taken in the medulla showing non-secreting cells (white arrows) that have internalized the 833D4-scFv. FIG. 10C shows fluorescence microscopy images showing the colocalization of the scFv with pS129-aSyn inside a neuron. FIG. 10D shows fluorescence microscopy images showing the internalization of the scFv by microglia. As shown, cells infected with the viral vectors effectively express the scFvs and secrete them in the brain. In addition, the secreted scFvs are internalized by neurons and microglia. Binding to extracellular and intracellular pS129-aSyn was observed.

These data suggest that both 833D4 and 73B3 were effective in preventing alpha-synuclein aggregation and alpha-synuclein fibril internalization. 833D4 and 73B3 were both found to be functionally superior to commercially available antibodies, such as Abcam ab209420, Wako (pSyn #64; FUJIFILM™ Wako Pure Chemical Corporation) and Abcam (ab51253). These data further suggest that both 833D4 and 73B3 are potential candidates for synucleinopathy therapies. These data also demonstrate that scFvs are effectively internalized by neurons and microglia and colocalize with extracellular and intracellular pS129-aSyn, thereby directly acting on pathological extracellular and intracellular alpha-synuclein aggregates (e.g., Lewy Bodies). Therefore, the antibodies defined herein can target both extracellular and intracellular aSyn aggregates, thereby enhancing their therapeutic potential.

### 833D4 and 73B3 AAV-scFvs Systemic Injection do not Cause Inflammation in Mice

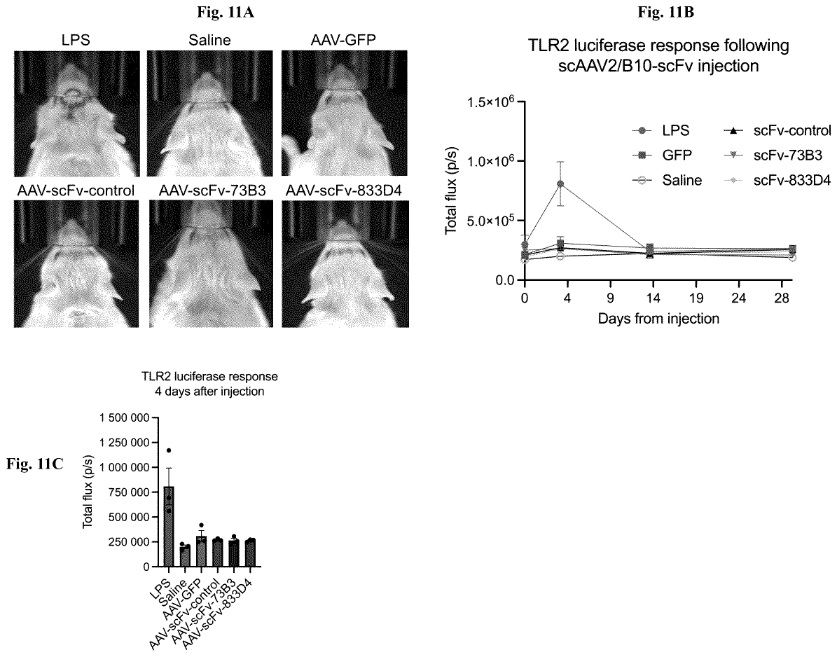

FIGS. 11A-11C show the safety results of AAV-scFv systemic injection. Transgenic mice expressing a Luciferase reporter under the transcriptional control of the TLR2 promoter were used. The expression of the TLR2 receptor is increased in inflammatory conditions. The level of luminescence is proportional to the level of TLR2, an indirect measure of inflammation. Mice were injected intravenously with LPS (positive control), saline, AAV-GFP, AAV-scFv-control, AAV-scFv-73B3 or AAV-scFv-833D4. FIG. 11A shows representative images of the bioluminescence signal in injected mice. FIG. 11B shows total flux (photon/second) of the bioluminescence signal in each group from baseline to 30 days post-injection. FIG. 11C shows total flux at 4 days post-injection. As shown in FIGS. 11A-11C, both 833D4 and 73B3 AAV-scFvs do not induce inflammation in mice upon systemic injection, as compared to LPS.

These data suggest that 833D4 and 73B3 are potentially safe and are potentially good candidates for synucleinopathy therapies and systemic administration.

### REFERENCES

Choi, J-H. et al. Optimization of AAV expression cassettes to improve packaging capacity and transgene expression in neurons. Mol Brain. 2014; 7: 17.
Luk, K. et al. Pathological α-synuclein transmission initiates Parkinson-like neurodegeneration in nontransgenic mice. Science. 2012 Nov. 16; 338(6109):949-53.
Mitra S, Tomar PC. Hybridoma technology; advancements, clinical significance, and future aspects. J Genet Eng Biotechnol. 2021 Oct. 18; 19(1):159.

### ITEMS

In some aspects, embodiments of the present disclosure as described herein include the following items:

1. An antibody or an antigen-binding fragment thereof that specifically binds to alpha-synuclein (aSyn), wherein the antibody or antigen-binding fragment thereof comprises:
a heavy chain variable region (VH) complementarity determining region 1 (CDR1), a VH CDR2, and a VH CDR3 from a VH comprising the amino acid sequence of SEQ ID NO: 5 or SEQ ID NO: 21; and
a light chain variable region (VL) CDR1, a VL CDR2, and a VL CDR3 from a VL comprising the amino acid sequence of SEQ ID NO: 6 or SEQ ID NO: 22.

2. The antibody or antigen-binding fragment thereof of item 1, comprising:
a VH CDR1, a VH CDR2, and a VH CDR3 from a VH comprising the amino acid sequence of SEQ ID NO: 5, and a VL CDR1, a VL CDR2, and a VL CDR3 from the VL comprising the amino acid sequence of SEQ ID NO: 6; or
a VH CDR1, a VH CDR2, and a VH CDR3 from a VH comprising the amino acid sequence of SEQ ID NO: 21, and a VL CDR1, a VL CDR2, and a VL CDR3 from the VL comprising the amino acid sequence of SEQ ID NO: 22.

3. The antibody or antigen-binding fragment thereof of item 1 or 2, comprising:
a VH CDR1 comprising the amino acid sequence of SEQ ID NO: 7, a VH CDR2 comprising the amino acid sequence of SEQ ID NO: 8, a VH CDR3 comprising the amino acid sequence of SEQ ID NO: 9, a VL CDR1 comprising the amino acid sequence of SEQ ID NO: 10, a VL CDR2 comprising the amino acid sequence of SEQ ID NO: 11, and a VL CDR3 comprising the amino acid sequence of SEQ ID NO: 12;
a VH CDR1 comprising the amino acid sequence of SEQ ID NO: 13, a VH CDR2 comprising the amino acid sequence of SEQ ID NO: 14, a VH CDR3 comprising the amino acid sequence of SEQ ID NO: 15, a VL CDR1 comprising the amino acid sequence of SEQ ID NO: 16, a VL CDR2 comprising the amino acid sequence RMS or the amino acid sequence of SEQ ID NO: 17, and a VL CDR3 comprising the amino acid sequence of SEQ ID NO: 18;
a VH CDR1 comprising the amino acid sequence of SEQ ID NO: 23, a VH CDR2 comprising the amino acid sequence of SEQ ID NO: 24, a VH CDR3 comprising the amino acid sequence of SEQ ID NO: 25, a VL CDR1 comprising the amino acid sequence of SEQ ID NO: 26, a VL CDR2 comprising the amino acid sequence of SEQ ID NO: 27, and a VL CDR3 comprising the amino acid sequence of SEQ ID NO: 28; or
a VH CDR1 comprising the amino acid sequence of SEQ ID NO: 29, a VH CDR2 comprising the amino acid sequence of SEQ ID NO: 30, a VH CDR3 comprising the amino acid sequence of SEQ ID NO: 31, a VL CDR1 comprising the amino acid sequence of SEQ ID NO: 32, a VL CDR2 comprising the amino acid sequence WAS or the amino acid sequence of SEQ ID NO: 33, and a VL CDR3 comprising the amino acid sequence of SEQ ID NO: 34.

4. The antibody or antigen-binding fragment thereof of any one of items 1 to 3, wherein the antibody or antigen-binding fragment thereof comprises a VH comprising an amino acid sequence that is at least about 70%, at least about 75%, at least about 80%, at least about 85%, at least about 90%, at least about 95%, at least about 96%, at least about 97%, at least about 98%, at least about 99% identical to SEQ ID NO: 5 or SEQ ID NO: 21.

5. The antibody or antigen-binding fragment thereof of any one of items 1 to 4, wherein the antibody or antigen-binding fragment thereof comprises a VL comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 6 or SEQ ID NO: 22.

6. The antibody or antigen-binding fragment thereof of any one of items 1 to 5, wherein the antibody or antigen-binding fragment thereof comprises a VH encoded by a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 3 or SEQ ID NO: 19.

7. The antibody or antigen-binding fragment thereof of any one of items 1 to 6, wherein the antibody or antigen-binding fragment thereof comprises a VL encoded by a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 4 or SEQ ID NO: 20.

8. The antibody or antigen-binding fragment thereof of any one of items 1 to 7, wherein the antibody or antigen-binding fragment thereof comprises a VH comprising the amino acid sequence of SEQ ID NO: 5 or SEQ ID NO: 21.

9. The antibody or antigen-binding fragment thereof of any one of items 1 to 8, wherein the antibody or antigen-binding fragment thereof comprises a VL comprising the amino acid sequence of SEQ ID NO: 6 or SEQ ID NO: 22.

10. The antibody or antigen-binding fragment thereof of any one of items 1 to 9, wherein the antibody or antigen-binding fragment thereof comprises a VH/VL pairing as set forth in SEQ ID NO: 5/SEQ ID NO: 6 or SEQ ID NO: 21/SEQ ID NO: 22.

11. The antibody or antigen-binding fragment thereof of any one of items 1 to 10, wherein the antibody or antigen-binding fragment thereof is from a human, primate, non-human primate (e.g., monkey), mouse, rat, rabbit, hamster, donkey, sheep, or cow.

12. The antibody or antigen-binding fragment thereof of any one of items 1 to 11, wherein the antibody has an IgG, IgD, IgE, IgM, or IgA isotype.

13. The antibody or antigen-binding fragment thereof of item 12, wherein the IgG antibody has an IgG1, IgG2, IgG2a/2c, IgG2b, IgG3, or IgG4 subclass.

14. The antibody or antigen-binding fragment thereof of any one of items 1 to 13, wherein the antibody or antigen-binding fragment thereof is selected from the group consisting of a scFv, scFv-Fc, minibody, monovalent IgG, diabody, triabody, tetrabody, Fab, Fab′, Fv, F(ab′)2, and any combination thereof.

15. The antibody or antigen-binding fragment thereof of any one of items 1 to 13, wherein the antibody or antigen-binding fragment thereof is selected from the group consisting of a kappa-lambda body, a dual-affinity re-targeting molecule (DART), a knob-in-hole, a strand-exchange engineered domain body (SEEDbody), a Bispecific T cell engager (BiTe), a CrossMab, an Fcab, a Diabody, a Tandem diabody (TandAb), and a DuoBody

16. The antibody or antigen-binding fragment thereof of any one of items 1 to 15, wherein the antibody or antigen-binding fragment thereof is humanized.

17. The antibody or antigen-binding fragment thereof of any one of items 1 to 16, wherein the antibody or antigen-binding fragment thereof specifically binds to phosphorylated alpha-synuclein (paSyn).

18. The antibody or antigen-binding fragment thereof of item 17, wherein the paSyn is phosphorylated at serine 129 of SEQ ID NO: 1 or at a residue corresponding to, positionally-equivalent to, or homologous to serine 129 of SEQ ID NO: 1.

19. An antibody or an antigen-binding fragment thereof that specifically binds to alpha-synuclein (aSyn), wherein the antibody or antigen-binding fragment thereof comprises:
a heavy chain variable region (VH) comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 5 or SEQ ID NO: 21; and
a light chain variable region (VL) comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 6 or SEQ ID NO: 22.

20. The antibody or antigen-binding fragment thereof of item 19, comprising the features of any one of items 1 to 18.

21. A polynucleotide encoding the antibody or antigen-binding fragment thereof of any one of items 1 to 20.

22. A vector comprising the polynucleotide of item 21.

23. The vector of item 22, which is a retroviral vector, a DNA vector, a plasmid, an RNA vector, an adenoviral vector, an adenovirus associated vector, a lentiviral vector, or any combination thereof.

24. A cell comprising the polynucleotide of item 21 or the vector of item 22 or 23.

25. The cell of item 24, wherein the cell is an immune cell.

26. The cell of item 24 or 25, wherein the cell is a chimeric antigen receptor (CAR) immune cell (e.g., CAR T cell or CAR NK cell).

27. A chimeric antigen receptor (CAR) comprising the antibody or antigen-binding fragment thereof of any one of items 1 to 20.

28. A polypeptide comprising the antibody or antigen-binding fragment thereof of any one of items 1 to 20.

29. An in vitro method of making a cell expressing the antibody or antigen-binding fragment thereof of any one of items 1 to 20 comprising transducing the cell with the polynucleotide of item 21 under suitable conditions.

30. The method of item 29, further comprising isolating the cell.

31. An in vitro method of making the antibody or antigen-binding fragment thereof of any one of items 1 to 20 comprising incubating the cell of item 24 under suitable conditions.

32. A pharmaceutical composition comprising the antibody or antigen-binding fragment thereof of any one of items 1 to 20, the vector of item 22 or 23, the cell of any one of items 24 to 26, the CAR of item 27, or the polypeptide of item 28, and a pharmaceutically acceptable carrier, excipient, diluent, solubilizer, emulsifier, preservative and/or adjuvant.

33. The antibody or antigen-binding fragment thereof of any one of items 1 to 20, the vector of item 22 or 23, the cell of any one of items 24 to 26, the CAR of item 27, the polypeptide of item 28, or the pharmaceutical composition of item 32, for use in:
the treatment of a synucleinopathy;
the detection, diagnosis, or prognosis of a synucleinopathy; or
the monitoring of the progression of a synucleinopathy;
   in a subject.

34. The antibody or antigen-binding fragment thereof, the vector, the cell, the CAR, the polypeptide, or the pharmaceutical composition for use of item 33, wherein the synucleinopathy is Parkinson's disease (PD), Lewy body disease (LBD), dementia with Lewy bodies, Multiple systemic atrophy (MSA), pure autonomic failure, or rapid eye movement (REM) sleep behaviour disorder.

35. Use of the antibody or antigen-binding fragment thereof of any one of items 1 to 20, the vector of item 22 or 23, the cell of any one of items 24 to 26, the CAR of item 27, the polypeptide of item 28, or the pharmaceutical composition of item 32 in:
the treatment of a synucleinopathy;
the detection, diagnosis, or prognosis of a synucleinopathy; or
the monitoring of the progression of a synucleinopathy;
   in a subject.

36. Use of the antibody or antigen-binding fragment thereof of any one of items 1 to 20, the vector of item 22 or 23, the cell of any one of items 24 to 26, the CAR of item 27, the polypeptide of item 28, or the pharmaceutical composition of item 32 in the manufacture of a medicament for the treatment of a synucleinopathy.

37. The use of item 35 or 36, wherein the synucleinopathy is Parkinson's disease (PD), Lewy body disease (LBD), dementia with Lewy bodies, Multiple systemic atrophy (MSA), pure autonomic failure, or rapid eye movement (REM) sleep behaviour disorder.

38. A method for treating a synucleinopathy in subject in need thereof, said method comprising administering the antibody or antigen-binding fragment thereof of any one of items 1 to 20, the vector of item 22 or 23, the cell of any one of items 24 to 26, the CAR of item 27, the polypeptide of item 28, or the pharmaceutical composition of item 32 in said subject.

39. The method of item 38, wherein the synucleinopathy is Parkinson's disease (PD), Lewy body disease (LBD), dementia with Lewy bodies, Multiple systemic atrophy (MSA), pure autonomic failure, or rapid eye movement (REM) sleep behaviour disorder.

40. A method for the detection, diagnosis, or prognosis of a synucleinopathy, or for the monitoring of the progression of a synucleinopathy, in a subject, said method comprising detecting alpha-synuclein in the subject or biological sample from said subject with the antibody or antigen-binding fragment thereof of any one of items 1 to 20.

41. The method of item 40, further comprising comparing the levels of aSyn or phosphorylated aSyn to a reference or control subject or sample from said reference or control subject.

42. The method of any one of items 40, comprising administering the antibody or antigen-binding fragment thereof in said subject

43. The method of any one of items 40 to 42, wherein the subject is a human subject that has or is suspected of having the synucleinopathy.

44. An in vitro method of detecting alpha-synuclein (aSyn) in a sample, said method comprising:
contacting the sample with the antibody or antigen-binding fragment thereof of any one of items 1 to 20 to form a complex between the antibody or antigen-binding fragment thereof and alpha-synuclein;
detecting the complex between the antibody or antigen-binding fragment thereof and alpha-synuclein; and
optionally, measuring the levels of the complex between the antibody or antigen-binding fragment thereof and alpha-synuclein.

45. The method of item 45, wherein the sample is a biological sample.

46. The method of item 44 or 45, wherein the sample is a biological sample from a human subject having or suspected of a synucleinopathy.

47. The method of any one of items 40 to 43 and 46, wherein the synucleinopathy is Parkinson's disease (PD), Lewy body disease (LBD), dementia with Lewy bodies, Multiple systemic atrophy (MSA), pure autonomic failure, or rapid eye movement (REM) sleep behaviour disorder.

48. The method of any one of items 40 to 47, wherein the aSyn is phosphorylated.

49. The method of any one of items 40 to 48, wherein the aSyn is phosphorylated at serine 129 of SEQ ID NO: 1 or at a residue corresponding to, positionally-equivalent to, or homologous to serine 129 of SEQ ID NO: 1.

1. An antibody or an antigen-binding fragment thereof that specifically binds to alpha-synuclein (aSyn), wherein the antibody or antigen-binding fragment thereof comprises:
a heavy chain variable region (VH) complementarity determining region 1 (CDR1), a VH CDR2, and a VH CDR3 from a VH comprising the amino acid sequence of SEQ ID NO: 5 or SEQ ID NO: 21; and
a light chain variable region (VL) CDR1, a VL CDR2, and a VL CDR3 from a VL comprising the amino acid sequence of SEQ ID NO: 6 or SEQ ID NO: 22.

2. The antibody or antigen-binding fragment thereof of claim 1, comprising:
a VH CDR1, a VH CDR2, and a VH CDR3 from a VH comprising the amino acid sequence of SEQ ID NO: 5, and a VL CDR1, a VL CDR2, and a VL CDR3 from the VL comprising the amino acid sequence of SEQ ID NO: 6; or
a VH CDR1, a VH CDR2, and a VH CDR3 from a VH comprising the amino acid sequence of SEQ ID NO: 21, and a VL CDR1, a VL CDR2, and a VL CDR3 from the VL comprising the amino acid sequence of SEQ ID NO: 22.

3. The antibody or antigen-binding fragment thereof of claim 1 or 2, comprising:
a VH CDR1 comprising the amino acid sequence of SEQ ID NO: 7, a VH CDR2 comprising the amino acid sequence of SEQ ID NO: 8, a VH CDR3 comprising the amino acid sequence of SEQ ID NO: 9, a VL CDR1 comprising the amino acid sequence of SEQ ID NO: 10, a VL CDR2 comprising the amino acid sequence of SEQ ID NO: 11, and a VL CDR3 comprising the amino acid sequence of SEQ ID NO: 12;
a VH CDR1 comprising the amino acid sequence of SEQ ID NO: 13, a VH CDR2 comprising the amino acid sequence of SEQ ID NO: 14, a VH CDR3 comprising the amino acid sequence of SEQ ID NO: 15, a VL CDR1 comprising the amino acid sequence of SEQ ID NO: 16, a VL CDR2 comprising the amino acid sequence RMS or the amino acid sequence of SEQ ID NO: 17, and a VL CDR3 comprising the amino acid sequence of SEQ ID NO: 18;
a VH CDR1 comprising the amino acid sequence of SEQ ID NO: 23, a VH CDR2 comprising the amino acid sequence of SEQ ID NO: 24, a VH CDR3 comprising the amino acid sequence of SEQ ID NO: 25, a VL CDR1 comprising the amino acid sequence of SEQ ID NO: 26, a VL CDR2 comprising the amino acid sequence of SEQ ID NO: 27, and a VL CDR3 comprising the amino acid sequence of SEQ ID NO: 28; or
a VH CDR1 comprising the amino acid sequence of SEQ ID NO: 29, a VH CDR2 comprising the amino acid sequence of SEQ ID NO: 30, a VH CDR3 comprising the amino acid sequence of SEQ ID NO: 31, a VL CDR1 comprising the amino acid sequence of SEQ ID NO: 32, a VL CDR2 comprising the amino acid sequence WAS or the amino acid sequence of SEQ ID NO: 33, and a VL CDR3 comprising the amino acid sequence of SEQ ID NO: 34.

4. The antibody or antigen-binding fragment thereof of any one of claims 1 to 3, wherein the antibody or antigen-binding fragment thereof comprises a VH comprising an amino acid sequence that is at least about 70%, at least about 75%, at least about 80%, at least about 85%, at least about 90%, at least about 95%, at least about 96%, at least about 97%, at least about 98%, at least about 99% identical to SEQ ID NO: 5 or SEQ ID NO: 21.

5. The antibody or antigen-binding fragment thereof of any one of claims 1 to 4, wherein the antibody or antigen-binding fragment thereof comprises a VL comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 6 or SEQ ID NO: 22.

6. The antibody or antigen-binding fragment thereof of any one of claims 1 to 5, wherein the antibody or antigen-binding fragment thereof comprises a VH encoded by a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 3 or SEQ ID NO: 19.

7. The antibody or antigen-binding fragment thereof of any one of claims 1 to 6, wherein the antibody or antigen-binding fragment thereof comprises a VL encoded by a nucleotide sequence that is at least about 70%, at least about 75%, at least about 80%, at least about 85%, at least about 90%, at least about 95%, at least about 96%, at least about 97%, at least about 98%, at least about 99%, or about 100% identical to SEQ ID NO: 4 or SEQ ID NO: 20.

8. The antibody or antigen-binding fragment thereof of any one of claims 1 to 7, wherein the antibody or antigen-binding fragment thereof comprises a VH comprising the amino acid sequence of SEQ ID NO: 5 or SEQ ID NO: 21.

9. The antibody or antigen-binding fragment thereof of any one of claims 1 to 8, wherein the antibody or antigen-binding fragment thereof comprises a VL comprising the amino acid sequence of SEQ ID NO: 6 or SEQ ID NO: 22.

10. The antibody or antigen-binding fragment thereof of any one of claims 1 to 9, wherein the antibody or antigen-binding fragment thereof comprises a VH/VL pairing as set forth in SEQ ID NO: 5/SEQ ID NO: 6 or SEQ ID NO: 21/SEQ ID NO: 22.

11. The antibody or antigen-binding fragment thereof of any one of claims 1 to 10, wherein the antibody or antigen-binding fragment thereof is from a human, primate, non-human primate (e.g., monkey), mouse, rat, rabbit, hamster, donkey, sheep, or cow.

12. The antibody or antigen-binding fragment thereof of any one of claims 1 to 11, wherein the antibody has an IgG, IgD, IgE, IgM, or IgA isotype.

13. The antibody or antigen-binding fragment thereof of claim 12, wherein the IgG antibody has an IgG1, IgG2, IgG2a/2c, IgG2b, IgG3, or IgG4 subclass.

14. The antibody or antigen-binding fragment thereof of any one of claims 1 to 13, wherein the antibody or antigen-binding fragment thereof is selected from the group consisting of a scFv, scFv-Fc, minibody, monovalent IgG, diabody, triabody, tetrabody, Fab, Fab′, Fv, F(ab′)2, and any combination thereof.

15. The antibody or antigen-binding fragment thereof of any one of claims 1 to 13, wherein the antibody or antigen-binding fragment thereof is selected from the group consisting of a kappa-lambda body, a dual-affinity re-targeting molecule (DART), a knob-in-hole, a strand-exchange engineered domain body (SEEDbody), a Bispecific T cell engager (BiTe), a CrossMab, an Fcab, a Diabody, a Tandem diabody (TandAb), and a DuoBody

16. The antibody or antigen-binding fragment thereof of any one of claims 1 to 15, wherein the antibody or antigen-binding fragment thereof is humanized.

17. The antibody or antigen-binding fragment thereof of any one of claims 1 to 16, wherein the antibody or antigen-binding fragment thereof specifically binds to phosphorylated alpha-synuclein (paSyn).

18. The antibody or antigen-binding fragment thereof of claim 17, wherein the paSyn is phosphorylated at serine 129 of SEQ ID NO: 1 or at a residue corresponding to, positionally-equivalent to, or homologous to serine 129 of SEQ ID NO: 1.

19. An antibody or an antigen-binding fragment thereof that specifically binds to alpha-synuclein (aSyn), wherein the antibody or antigen-binding fragment thereof comprises:
a heavy chain variable region (VH) comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 5 or SEQ ID NO: 21; and
a light chain variable region (VL) comprising an amino acid sequence that is at least about 70%, at least about 75%, at least about 80%, at least about 85%, at least about 90%, at least about 95%, at least about 96%, at least about 97%, at least about 98%, or at least about 99% identical to SEQ ID NO: 6 or SEQ ID NO: 22.

20. The antibody or antigen-binding fragment thereof of claim 19, comprising the features of any one of claims 1 to 18.

21. A polynucleotide encoding the antibody or antigen-binding fragment thereof of any one of claims 1 to 20.

22. A vector comprising the polynucleotide of claim 21.

23. The vector of claim 22, which is a retroviral vector, a DNA vector, a plasmid, an RNA vector, an adenoviral vector, an adenovirus associated vector, a lentiviral vector, or any combination thereof.

24. A cell comprising the polynucleotide of claim 21 or the vector of claim 22 or 23.

25. The cell of claim 24, wherein the cell is an immune cell.

26. The cell of claim 24 or 25, wherein the cell is a chimeric antigen receptor (CAR) immune cell (e.g., CAR T cell or CAR NK cell).

27. A chimeric antigen receptor (CAR) comprising the antibody or antigen-binding fragment thereof of any one of claims 1 to 20.

28. A polypeptide comprising the antibody or antigen-binding fragment thereof of any one of claims 1 to 20.

29. An in vitro method of making a cell expressing the antibody or antigen-binding fragment thereof of any one of claims 1 to 20 comprising transducing the cell with the polynucleotide of claim 21 under suitable conditions.

30. The method of claim 29, further comprising isolating the cell.

31. An in vitro method of making the antibody or antigen-binding fragment thereof of any one of claims 1 to 20 comprising incubating the cell of claim 24 under suitable conditions.

32. A pharmaceutical composition comprising the antibody or antigen-binding fragment thereof of any one of claims 1 to 20, the vector of claim 22 or 23, the cell of any one of claims 24 to 26, the CAR of claim 27, or the polypeptide of claim 28, and a pharmaceutically acceptable carrier, excipient, diluent, solubilizer, emulsifier, preservative and/or adjuvant.

33. The antibody or antigen-binding fragment thereof of any one of claims 1 to 20, the vector of claim 22 or 23, the cell of any one of claims 24 to 26, the CAR of claim 27, the polypeptide of claim 28, or the pharmaceutical composition of claim 32, for use in:
the treatment of a synucleinopathy;
the detection, diagnosis, or prognosis of a synucleinopathy; or
the monitoring of the progression of a synucleinopathy;
in a subject.

34. The antibody or antigen-binding fragment thereof, the vector, the cell, the CAR, the polypeptide, or the pharmaceutical composition for use of claim 33, wherein the synucleinopathy is Parkinson's disease (PD), Lewy body disease (LBD), dementia with Lewy bodies, Multiple systemic atrophy (MSA), pure autonomic failure, or rapid eye movement (REM) sleep behaviour disorder.

35. Use of the antibody or antigen-binding fragment thereof of any one of claims 1 to 20, the vector of claim 22 or 23, the cell of any one of claims 24 to 26, the CAR of claim 27, the polypeptide of claim 28, or the pharmaceutical composition of claim 32 in:
the treatment of a synucleinopathy;
the detection, diagnosis, or prognosis of a synucleinopathy; or
the monitoring of the progression of a synucleinopathy;
in a subject.

36. Use of the antibody or antigen-binding fragment thereof of any one of claims 1 to 20, the vector of claim 22 or 23, the cell of any one of claims 24 to 26, the CAR of claim 27, the polypeptide of claim 28, or the pharmaceutical composition of claim 32 in the manufacture of a medicament for the treatment of a synucleinopathy.

37. The use of claim 35 or 36, wherein the synucleinopathy is Parkinson's disease (PD), Lewy body disease (LBD), dementia with Lewy bodies, Multiple systemic atrophy (MSA), pure autonomic failure, or rapid eye movement (REM) sleep behaviour disorder.

38. A method for treating a synucleinopathy in subject in need thereof, said method comprising administering the antibody or antigen-binding fragment thereof of any one of claims 1 to 20, the vector of claim 22 or 23, the cell of any one of claims 24 to 26, the CAR of claim 27, the polypeptide of claim 28, or the pharmaceutical composition of claim 32 in said subject.

39. The method of claim 38, wherein the synucleinopathy is Parkinson's disease (PD), Lewy body disease (LBD), dementia with Lewy bodies, Multiple systemic atrophy (MSA), pure autonomic failure, or rapid eye movement (REM) sleep behaviour disorder.

40. A method for the detection, diagnosis, or prognosis of a synucleinopathy, or for the monitoring of the progression of a synucleinopathy, in a subject, said method comprising detecting alpha-synuclein in the subject or biological sample from said subject with the antibody or antigen-binding fragment thereof of any one of claims 1 to 20.

41. The method of claim 40, further comprising comparing the levels of aSyn or phosphorylated aSyn to a reference or control subject or sample from said reference or control subject.

42. The method of any one of claim 40, comprising administering the antibody or antigen-binding fragment thereof in said subject

43. The method of any one of claims 40 to 42, wherein the subject is a human subject that has or is suspected of having the synucleinopathy.

44. An in vitro method of detecting alpha-synuclein (aSyn) in a sample, said method comprising:
contacting the sample with the antibody or antigen-binding fragment thereof of any one of claims 1 to 20 to form a complex between the antibody or antigen-binding fragment thereof and alpha-synuclein;
detecting the complex between the antibody or antigen-binding fragment thereof and alpha-synuclein; and
optionally, measuring the levels of the complex between the antibody or antigen-binding fragment thereof and alpha-synuclein.

45. The method of claim 45, wherein the sample is a biological sample.

46. The method of claim 44 or 45, wherein the sample is a biological sample from a human subject having or suspected of a synucleinopathy.

47. The method of any one of claims 40 to 43 and 46, wherein the synucleinopathy is Parkinson's disease (PD), Lewy body disease (LBD), dementia with Lewy bodies, Multiple systemic atrophy (MSA), pure autonomic failure, or rapid eye movement (REM) sleep behaviour disorder.

48. The method of any one of claims 40 to 47, wherein the aSyn is phosphorylated.

49. The method of any one of claims 40 to 48, wherein the aSyn is phosphorylated at serine 129 of SEQ ID NO: 1 or at a residue corresponding to, positionally-equivalent to, or homologous to serine 129 of SEQ ID NO: 1.
